(19) United States
(12) Patent Application Publication
(10) Pub. No.: US 2017/0007720 A1
(43) Pub. Date: Jan. 12, 2017

(54) METHODS AND COMPOSITIONS FOR GENE DELIVERY TO ON BIPOLAR CELLS

(71) Applicant: UNIVERSITY OF FLORIDA RESEARCH FOUNDATION, INC., Gainesville, FL (US)

(72) Inventors: Shannon E. Boye, Gainesville, FL (US); Frank Dyka, Gainesville, FL (US); Sanford L. Boye, Gainesville, FL (US); William W. Hauswirth, Gainesville, FL (US)

(73) Assignee: University of Florida Research Foundation, Inc., Gainesville, FL (US)

(21) Appl. No.: 15/120,425

(22) PCT Filed: Feb. 18, 2015
(86) PCT No.: PCT/US15/16424

§ 371 Date: Aug. 19, 2016

Related U.S. Application Data
(60) Provisional application No. 61/943,154, filed on Feb. 21, 2014.

(51) Int. Cl. A61K 48/00; C12N 15/86; C12N 7/00; A61K 45/06; A61K 38/17

(52) U.S. Cl. CPC A61K 48/0058; A61K 48/0075; A61K 48/0083; A61K 45/06; A61K 38/1709; C12N 7/00; C12N 15/86; C12N 2750/14122; C12N 2750/14123; C12N 2750/14143; C12N 2750/14171; C12N 2750/14145; C12N 2830/008

(57) ABSTRACT

Disclosed are capsid-modified rAAV expression vectors, as well as infectious virions, compositions, and pharmaceutical formulations that include them. Also disclosed are methods of preparing and using novel capsid-protein-mutated rAAV vector constructs in a variety of diagnostic and therapeutic applications including, inter alia, as delivery agents for diagnosis, treatment, or amelioration of one or more diseases, disorders, or dysfunctions of the mammalian eye. Also disclosed are methods for intravitreal delivery of therapeutic gene constructs to retinal neuron cells, and specifically to ON bipolar cells, of the mammalian eye, as well as use of the disclosed compositions in the manufacture of medicaments for a variety of in vitro and/or in vivo applications including the treatment of retinitis pigmentosa, melanoma-associated retinopathy, and congenital stationary night blindness.

### CROSS-REFERENCE TO RELATED APPLICATIONS

This application claims the benefit of the filing date of U.S. Provisional Application No. 61/943,154 filed Feb. 21, 2014, the entire contents of which are incorporated by reference herein.

### FIELD OF THE INVENTION

The present invention relates generally to the fields of molecular biology and virology, and in particular, to the development of gene delivery vehicles.

### BACKGROUND OF THE INVENTION

Major advances in the field of gene therapy have been achieved by using viruses to deliver therapeutic genetic material. The adeno-associated virus (AAV) has attracted considerable attention as a highly effective viral vector for gene therapy due to its low immunogenicity and ability to effectively transduce non-dividing cells. AAV has been shown to infect a variety of cell and tissue types, and significant progress has been made over the last decade to adapt this viral system for use in human gene therapy.

In its normal “wild type” form, AAV (AAV) DNA is packaged into the viral capsid as a single-stranded molecule about 4600 nucleotides (nt) in length. Following infection of the cell by the virus, the molecular machinery of the cell converts the single-stranded DNA into a double-stranded form. Only this double-stranded DNA form is able to be transcribed by cellular enzymes into RNA, which is then translated into polypeptides by additional cellular pathways.

AAV has many properties that favor its use as a gene delivery vehicle: 1) the wild-type virus is not associated with any pathologic human condition; 2) the recombinant AAV (rAAV) form does not contain native viral coding sequences; and 3) persistent transgenic expression has been observed in a variety of mammalian cells, facilitating their use in many gene therapy-based applications.

The transduction efficiency of rAAV2 vectors varies greatly in different cells and tissues in vitro and in vivo, and that fact limits their usefulness in certain gene therapy regimens. What is lacking in the prior art are improved rAAV viral vectors that have enhanced transduction efficiency for infecting selected mammalian cells, and for targeted gene delivery to human cells in particular.

Leber Congenital Amaurosis

Clinical trials for RPE65-Leber congenital amaurosis (LCA) have demonstrated the ability to deliver therapeutic transgene to the retinal pigment epithelium (RPE) by subretinal injection thereby restoring retinal function and visually-evoked behavior to patients (Cideciyan et al., 2009; Maguire et al., 2008; Bainbridge et al., 2008). Given the predominance of photoreceptor (PR) specific retinal degenerations (Wright et al., 2010), there is a need to develop PR targeted gene therapies.

What is lacking in the prior art are viral vectors that are capable of transducing retinal cells (e.g., photoreceptors or bipolar cells), and methods for using them that are less invasive than conventional sub-retinal injection protocols. The development of such vectors, and compositions comprising them would provide a major advancement in retinal gene therapy.

### BRIEF SUMMARY OF THE INVENTION

The present disclosure overcomes these and other limitations inherent in the prior art by providing novel AAV vectors that are capable of, and optimized for, transducing photoreceptors following ocular (e.g., intravitreal) delivery. The disclosure also provides a robust methodology for detecting and quantifying photoreceptor transduction in vivo.

Advantageously, the novel rAAV vectors, expression constructs, and infectious virions and viral particles comprising them as disclosed herein preferably have an improved efficiency in transducing one or more of retinal cells of a mammalian eye, and in particular, one or more ON bipolar cells of a human eye.

The improved rAAV vectors provided hererin preferably transduce the one or more mammalian retinal cells at higher-efficiencies (and often, much higher efficiencies) than conventional, wild-type, unmodified rAAV vector constructs. By employing multi-mutated capsid protein-encoding rAAV vectors (including those having combinations of three, four, five, or even more surface-exposed amino acid residues) from a variety of AAV serotypes, the inventors have developed a collection of multi-mutated rAAV vectors containing ON bipolar cell-specific promoters operably linked to a nucleic acid segment that encodes one or more therapeutic agents. The novel vector constructs have improved properties and are capable of higher-efficiency transduction than the corresponding, non-substituted (i.e., un-modified) parent vectors from which the mutants were prepared.

In some aspects, the disclosure relates to an adeno-associated viral (AAV) particle comprising (a) a recombinant adeno-associated viral (rAAV) vector polynucleotide that comprises a nucleic acid segment that encodes a diagnostic or therapeutic agent operably linked to an ON bipolar cell-specific promoter that is capable of expressing the nucleic acid segment in one or more middle retinal neuron cells of a mammalian eye; and (b) a modified capsid protein, wherein the modified capsid protein comprises at least a first non-native amino acid at a position that corresponds to a surface-exposed amino acid residue in the wild-type AAV2 capsid protein, and further wherein the transduction efficiency (e.g., in ON bipolar cells) of a virion comprising the modified capsid protein is higher than that of a virion comprising a corresponding, unmodified wild-type capsid protein.

In some embodiments, the modified capsid protein comprises three or more non-native amino acid substitutions at positions corresponding to three distinct surface-exposed amino acid residues of the wild-type AAV2 capsid protein (e.g., as set forth in SEQ ID NO:2); or to three distinct surface-exposed amino acid residues corresponding thereto in any one of the wild-type AAV1, AAV3, AAV4, AAV5, AAV6, AAV7, AAV9, or AAV10 capsid proteins (e.g., as set forth, respectively, in SEQ ID NO:1, SEQ ID NO:3, SEQ ID NO:4, SEQ ID NO:5, SEQ ID NO:6, SEQ ID NO:7, SEQ ID NO:9, or SEQ ID NO:10), or any combination thereof.

In some embodiments, the non-native amino acid substitutions occur at amino acid residues:(a) Y272, Y444, Y500, and Y730; (b) Y272, Y444, Y500, Y700, and Y730; (c) Y272, Y444, Y500, Y704, and Y730; (d) Y252, Y272, Y444, Y500, Y704, and Y730; (e) Y272, Y444, Y500, Y700, Y704, and Y730; (f) Y252, Y272, Y444, Y500, Y700, Y704, and Y730; (g) Y444, Y500, Y730, and T491; (h) Y444, Y500, Y730, and S458; (i) Y444, Y500, Y730, S662, and T491; (j) Y444, Y500, Y730, T550, and T491; (k) Y444, Y500, Y730, T659, and T491; or (1) Y272, Y444, Y500, Y730, and T491 of the wild-type AAV2 capsid protein (e.g., as set forth in SEQ ID NO:2), or at equivalent amino acid positions corresponding thereto in any one of the wild-type AAV1, AAV3, AAV4, AAV5, AAV6, AAV7, AAV9, or AAV10 capsid proteins (e.g., as set forth, respectively, in SEQ ID NO:1, SEQ ID NO:3, SEQ ID NO:4, SEQ ID NO:5, SEQ ID NO:6, SEQ ID NO:7, SEQ ID NO:9, or SEQ ID NO:10), or any combination thereof.

In some embodiments, the AAV particle comprise the amino acid substitutions Y272F, Y444F, Y500F, Y730F, and T491V in a wild-type AAV2 capsid protein (e.g., as set forth in SEQ ID NO:2), or equivalent amino acid substitutions at the corresponding residues in any one of the wild-type AAV1, AAV3, AAV4, AAV5, AAV6, AAV7, AAV9, or AAV10 capsid proteins.

In some embodiments, the transduction efficiency of a virion comprising the modified capsid protein is about 2- to about 50-fold higher in the one or more middle retinal neuron cells than that of a virion that comprises a corresponding, unmodified, wild-type capsid protein (e.g., in ON bipolar cells).

In some embodiments, the nucleic acid segment further comprises an enhancer, a post-transcriptional regulatory sequence, a polyadenylation signal, or any combination thereof, operably linked to the nucleic acid segment encoding the diagnostic or therapeutic agent.

In some embodiments, the ON Bipolar cell-specific promoter is obtained from a mammalian purkinje cell protein 2 (PCP2) regulatory region. In some embodiments, the ON Bipolar cell-specific promoter obtained from the mammalian purkinje cell protein 2 (PCP2) regulatory region comprises or consists of the nucleotide sequence of SEQ ID NO: 12. Other exemplary ON Bipolar cell-specific promoters include a Grm6 promoter or a Grm6/SV40 promoter (see, e.g., Doroudchi et al. Virally delivered channelrhodopsin-2 safely and effectively restores visual function in multiple mouse models of blindness. Mol Ther. 2011 July; 19(7):1220-9; Lagali et al. Light-activated channels targeted to ON bipolar cells restore visual function in retinal degeneration. Nat Neurosci. 2008 June; 11(6):667-75; Cronin et al. Efficient transduction and optogenetic stimulation of retinal bipolar cells by a synthetic adeno-associated virus capsid and promoter. EMBO Mol Med. 2014 Aug. 4; 6(9):1175-90; and Gaub et al. Restoration of visual function by expression of a light-gated mammalian ion channel in retinal ganglion cells or ON-bipolar cells. Proc Natl Acad Sci USA. 2014 Dec. 23; 111(51):E5574-83.).

In some embodiments, the therapeutic agent is a polypeptide, a peptide, a ribozyme, a peptide nucleic acid, an siRNA, an RNAi, an antisense oligonucleotide, an antisense polynucleotide, an antibody, an antigen binding fragment, or any combination thereof.

In some embodiments, the therapeutic agent a Nyx polypeptide. In some embodiments, the Nyx polypeptide comprises or consists of an amino acid sequence that is encoded by the nucleotide sequence of SEQ ID NO: 13 or SEQ ID NO: 14.

Other aspects of the disclosure relate to a method for providing a mammal in need thereof with a therapeutically-effective amount of a selected therapeutic agent, the method comprising ocularly (e.g., intravitreally) administering to one or both eyes of the mammal, an amount of the AAV particle of any one of the embodiments above or described herein; and for a time effective to provide the mammal with a therapeutically-effective amount of the selected therapeutic agent.

Yet other aspects of the disclosure relate to a method for treating or ameliorating one or more symptoms of a disease, a disorder, a dysfunction, an injury, an abnormal condition, or trauma in a mammal, the method comprising, ocularly (e.g., intravitreally) administering to one or both eyes of the mammal in need thereof, the AAV particle of any one of the embodiments above or described herein, in an amount and for a time sufficient to treat or ameliorate the one or more symptoms of the disease, the disorder, the dysfunction, the injury, the abnormal condition, or the trauma in the mammal.

Other aspects of the disclosure relate to a method for expressing a nucleic acid segment in one or more retinal cells of a mammal, the method comprising: ocularly (e.g., intravitreally) administering to one or both eyes of the mammal the AAV particle of any one of the embodiments above or described herein, for a time effective to produce the therapeutic agent in the one or more retinal cells of the mammal.

In some embodiments, the mammal has, is suspected of having, is at risk for developing, or has been diagnosed with at least a first retinal disorder, a first retinal disease, or a first retinal dystrophy, or any combination thereof. In some embodiments, the retinal disease or disorder is retinitis pigmentosa, melanoma-associated retinopathy, congenital stationary night blindness, cone-rod dystrophy, Leber congenital amaurosis, or late stage age-related macular degeneration. In some embodiments, the mammal is a neonate, a newborn, an infant, or a juvenile. In some embodiments, the mammal is human.

In some embodiments, production of the therapeutic agent a) preserves one or more ON bipolar cells, b) restores one or more rod- and/or cone-mediated functions, c) restores visual behavior in one or both eyes, or d) any combination thereof. In some embodiments, production of the therapeutic agent persists in the one or more retinal cells substantially for a period of at least three months following a single intravitreal administration of the AAV particle into the one or both eyes of the mammal. In some embodiments, production of the therapeutic agent persists in the one or more retinal cells substantially for a period of at least six months following a single intravitreal administration.

In some embodiments, the rAAV vector polynucleotide comprised within the AAV particle is a self-complementary rAAV (scAAV).

In some embodiments, the therapeutic agent is an agonist, an antagonist, an anti-apoptosis factor, an inhibitor, a receptor, a cytokine, a cytotoxin, an erythropoietic agent, a glycoprotein, a growth factor, a growth factor receptor, a hormone, a hormone receptor, an interferon, an interleukin, an interleukin receptor, a nerve growth factor, a neuroactive peptide, a neuroactive peptide receptor, a protease, a protease inhibitor, a protein decarboxylase, a protein kinase, a protein kinsase inhibitor, an enzyme, a receptor binding protein, a transport protein or an inhibitor thereof, a serotonin receptor, or an uptake inhibitor thereof, a serpin, a serpin receptor, a tumor suppressor, a chemotherapeutic, or any combination thereof. In some embodiments, the therapeutic agent a Nyx polypeptide. In some embodiments, the Nyx polypeptide comprises or consists of an amino acid sequence that is encoded by the nucleotide sequence of SEQ ID NO: 13 or SEQ ID NO: 14.

Yet other aspects of the disclosure relate to a recombinant adeno-associated viral (rAAV) vector polynucleotide that comprises a nucleic acid segment that encodes a therapeutic agent operably linked to an ON bipolar cell-specific promoter that is capable of expressing the nucleic acid segment in one or more middle retinal neuron cells of a mammalian eye. In some embodiments, the ON Bipolar cell-specific promoter is obtained from a mammalian purkinje cell protein 2 (PCP2) regulatory region. In some embodiments, the ON Bipolar cell-specific promoter obtained from the mammalian purkinje cell protein 2 (PCP2) regulatory region comprises or consists of the nucleotide sequence of SEQ ID NO: 12. In some embodiments, the therapeutic agent a Nyx polypeptide. In some embodiments, the Nyx polypeptide comprises or consists of an amino acid sequence that is encoded by the nucleotide sequence of SEQ ID NO: 13 or SEQ ID NO: 14.

Other aspects of the disclosure relate to an adeno-associated viral (AAV) particle comprising: a modified capsid protein, wherein the modified capsid protein comprises non-native amino acid substitutions occur at amino acid residues Y272, Y444, Y500, Y730, and T491 in a wild-type AAV2 capsid protein (e.g., as set forth in SEQ ID NO:2), or equivalent amino acid substitutions at the corresponding residues in any one of the wild-type AAV1, AAV3, AAV4, AAV5, AAV6, AAV7, AAV9, or AAV10 capsid proteins. In some embodiments, the AAV particle comprises the amino acid substitutions Y272F, Y444F, Y500F, Y730F, and T491V in a wild-type AAV2 capsid protein (e.g., as set forth in SEQ ID NO:2), or equivalent amino acid substitutions at the corresponding residues in any one of the wild-type AAV1, AAV3, AAV4, AAV5, AAV6, AAV7, AAV9, or AAV10 capsid proteins. In some embodiments, the transduction efficiency of a virion comprising the modified capsid protein is about 2- to about 50-fold higher in the one or more middle retinal neuron cells than that of a virion that comprises a corresponding, unmodified, wild-type capsid protein.

Other aspects of the disclosure relate to a nucleic acid that encodes a modified capsid protein, wherein the modified capsid protein comprises non-native amino acid substitutions occur at amino acid residues Y272, Y444, Y500, Y730, and T491 in a wild-type AAV2 capsid protein (e.g., as set forth in SEQ ID NO:2), or equivalent amino acid substitutions at the corresponding residues in any one of the wild-type AAV1, AAV3, AAV4, AAV5, AAV6, AAV7, AAV9, or AAV10 capsid proteins. In some embodiments, the modified capsid protein comprises the amino acid substitutions Y272F, Y444F, Y500F, Y730F, and T491V in a wild-type AAV2 capsid protein (e.g., as set forth in SEQ ID NO:2), or equivalent amino acid substitutions at the corresponding residues in any one of the wild-type AAV1, AAV3, AAV4, AAV5, AAV6, AAV7, AAV9, or AAV10 capsid proteins. In some embodiments, the transduction efficiency of a virion comprising the modified capsid protein is about 2- to about 50-fold higher in the one or more middle retinal neuron cells than that of a virion that comprises a corresponding, unmodified, wild-type capsid protein.

Also disclosed are improved rAAV vector compositions useful in delivering a variety of nucleic acid segments, including those encoding therapeutic proteins polypeptides, peptides, antisense oligonucleotides, and ribozyme constructs to selected host cells for use in various diagnostic and/or therapeutic regimens. Methods are also provided for preparing and using these modified rAAV-based vector constructs in a variety of viral-based gene therapies, and in particular, for the diagnosis, prevention, treatment and/or amelioration of symptoms of visual defect and/or blindness. The disclosure also provides mutated rAAV-based viral vector delivery systems with increased transduction efficiency and/or improved viral infectivity of mammalian ON bipolar cells. In particular, the disclosure provides novel AAV capsid mutant/cellular promoter combination that, when delivered intravitreally, is capable of selectively driving transgene expression in retinal ON bipolar cells.

In a particular embodiment the disclosure provides improved rAAV vectors that have been derived from a number of different serotypes, including, for example, those selected from the group consisting of AAV1, AAV3, AAV4, AAV5, AAV7, AAV8, AAV9, and AAV10, whose capsid protein sequences are set forth in SEQ ID NO:1, SEQ ID NO:3, SEQ ID NO:4, SEQ ID NO:5, SEQ ID NO:7, SEQ ID NO:8, SEQ ID NO:9, and SEQ ID NO:10, respectively.

Exemplary multi-mutated VP3 capsid protein modified rAAV vectors of the present disclosure include, but are not limited to, those comprising three or more non-native amino acid substitutions at three or more amino acid residues selected from the group consisting of: (a) Y272, Y444, Y500, and Y730; (b) Y272, Y444, Y500, Y700, and Y730; (c) Y272, Y444, Y500, Y704, and Y730; (d) Y252, Y272, Y444, Y500, Y704, and Y730; (e) Y272, Y444, Y500, Y700, Y704, and Y730; (f) Y252, Y272, Y444, Y500, Y700, Y704, and Y730; (g) Y444, Y500, Y730, and T491; (h) Y444, Y500, Y730, and S458; (i) Y444, Y500, Y730, S662, and T491; (j) Y444, Y500, Y730, T550, and T491; (k) Y444, Y500, Y730, T659, and T491; and (1) Y272, Y444, Y500, Y730 and T491 of the wild-type AAV2 capsid protein as set forth in SEQ ID NO:2, or at equivalent amino acid positions corresponding thereto in any one of the wild-type AAV1, AAV3, AAV4, AAVS, AAV6, AAV7, AAV9, or AAV10 capsid proteins, as set forth, respectively, in SEQ ID NO:1, SEQ ID NO:3, SEQ ID NO:4, SEQ ID NO:5, SEQ ID NO:6, SEQ ID NO:7, SEQ ID NO:9, or SEQ ID NO:10, or any combination thereof.

The disclosure also provides an isolated and purified polynucleotide that encodes one or more of the disclosed rAAV vectors described herein, as well as pluralities of infectious adeno-associated viral virions that contain such a polynucleotide. Preferably, the vector constructs of the present disclosure further include at least one nucleic acid segment that encodes at least one ocular therapeutic agent operably linked to an ON bipolar cell-specific promoter that is capable of expressing the nucleic acid segment in suitable mammalian retinal cells that have been transformed with the vector construct. In some embodiments, an ON bipolar cell-specific promoter is a promoter that, when injected into the retina, results in expression of a target molecule (e.g., a therapeutic or diagnostic agent) preferentially or exclusivey in ON bipolar cells.

In the practice of the disclosure, the transduction efficiency of a virion comprising a multi-mutated, VP3 capsid protein-modified rAAV vector will be higher than that of the corresponding, unmodified, wild-type protein, and as such, will preferably possess a transduction efficiency in mammalian retinal cells that is at least 2-fold, at least about 4-fold, at least about 6-fold, at least about 8-fold, at least about 10-fold, or at least about 12-fold or higher in a selected mammalian host cell than that of a virion that comprises a corresponding, unmodified, capsid protein. In certain embodiments, the transduction efficiency of the rAAV vectors provided herein will be at least about 15-fold higher, at least about 20-fold higher, at least about 25-fold higher, at least about 30-fold higher, or at least about 40, 45, or 50-fold or more greater than that of a virion that comprises a corresponding, unmodified, capsid protein. In some embodiments, the transduction efficiency is evaluated in ON bipolar cells. Trasduction efficiency can be measured using any method known in the art or described herein, e.g., by PCR, immunofluorescence (e.g., if the rAAV vector encodes a fluorescent protein), and the like. Moreover, the infectious virions of the present disclosure that include one or more modified AAV VP3 capsid proteins are preferably less susceptible to ubiquitination when introduced into a mammalian cell than that of a virion that comprises a corresponding, unmodified, capsid protein.

The present disclosure also concerns rAAV vectors, wherein the nucleic acid segment further comprises a promoter, an enhancer, a post-transcriptional regulatory sequence, a polyadenylation signal, or any combination thereof, operably linked to the nucleic acid segment that encodes the selected polynucleotide of interest.

Preferably, the promoter is a heterologous promoter, and in particular, a mammalian ON bipolar-specific promoter such as the one described herein obtained from the purkinje cell protein 2 regulatory region.

In certain embodiments, the nucleic acid segments cloned into the novel rAAV expression vectors described herein will express or encode one or more polypeptides, peptides, ribozymes, peptide nucleic acids, siRNA' s, RNAi' s, antisense oligonucleotides, antisense polynucleotides, antibodies, antigen binding fragments, or any combination thereof.

As noted herein, the therapeutic agents useful in the disclosure may include one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof. In some embodiments, the therapeutic agent a Nyx polypeptide. In some embodiments, the Nyx polypeptide comprises or consists of an amino acid sequence that is encoded by the nucleotide sequence of SEQ ID NO: 13 or SEQ ID NO: 14. SEQ ID NO: 13 provides an exemplary human NYX cDNA. SEQ ID NO: 14 provies an exemplary mouse NYX cDNA.

Exemplary Human NYX cDNA

(SEQ ID NO: 13)
atgaaaggccgagggatgttggtcctgcttctgcatgcggtggtcctcgg
cctgcccagcgcctgggccgtgggggcctgcgcccgcgcttgtcccgccg
cctgcgcctgcagcaccgtggagcgcggctgctcggtgcgctgcgaccgc
gcgggcctcctgcgggtgccggccgagctcccgtgcgaggcggtctccat
cgacctggaccggaacggcctgcgcttcctgggcgagcgagccttcggca
cgctgccgtccttgcgccgcctgtcgctgcgccacaacaacctgtccttc
atcacgcccggcgccttcaagggcctgccgcgcctggctgagctgcgcct
ggcgcacaacggcgacctgcgctacctgcacgcgcgcaccttcgcggcgc
tcagccgcctgcgccgcctagacctagcagcctgccgcctcttcagcgtg
cccgagcgcctcctggccgaactgccggccctgcgcgaactcgccgcctt
cgacaacctgttccgccgcgtgccgggcgcgctgcgcggcctggccaacc
tgacgcacgcgcacctggagcgcggccgcatcgaggcggtggcctccagc
tcgctgcagggcctgcgccgcctgcgctcgctcagcctgcaggccaaccg
cgtccgtgccgtgcacgctggcgccttcggggactgtggcgtcctggagc
atctgctgctcaacgacaacctgctggccgagctcccggccgacgccttc
cgcggcctgcggcgcctgcgcacgctcaacctgggtggcaacgcgctgga
ccgcgtggcgcgcgcctggttcgctgacctggccgagctcgagctgctct
acctggaccgcaacagcatcgccttcgtggaggagggcgccttccagaac
ctctcgggtctcctcgcgctgcacctcaacggcaaccgcctcaccgtgct
cgcctgggtcgccttccagcccggcttcttcctgggccgcctcttcctct
tccgcaacccgtggtgctgcgactgccgtctggagtggctgagggactgg
atggagggctccggacgtgtcaccgacgtgccgtgcgcctccccgggctc
cgtggccggcctggacctcagccaggtgaccttcgggcgctcctccgatg
gcctctgtgtggaccccgaggagctgaacctcaccacgtccagtccaggc
ccgtccccagaaccagcggccaccaccgtgagcaggttcagcagcctcct
ctccaagctgctggccccgagggtcccggtggaggaggcggccaacacca
ctggggggctggccaacgcctccctgtccgacagcctctcctcccgtggg
gtgggaggcgcgggccggcagccctggtttctcctcgcctcttgtctcct
gcccagcgtggcccagcacgtggtgtttggcctgcagatggactga

Exemplary Mouse NYX cDNA

(SEQ ID NO: 14)
atgctgatcctgcttcttcatgcggtggtcttcagtctgccctacaccag
ggccaccgaggcctgtctgcgggcctgccctgcggcctgcacctgcagcc
acgtggaacgtggctgctcagtgcgctgtgaccgtgcgggcctccagcgg
gtgccccaggagtttccgtgcgaggcggcctccatcgatctggaccggaa
tggcctgcgcatcctgggcgagcgggcctttggcacgctgccgtcgttgc
gccgcctgtcgctgcgccacaataacctgtccttcatcacgcccggcgcc
ttcaagggcctgccgcggttggccgagctgcgcctggcgcacaacggtga
gctgcgctacctgcacgtgcggaccttcgcggcgctgggccgcctacgcc
gcctggacctggcggcctgccgcctcttcagcgtccccgagcgtctcctg
gccgagctgccggccctgcgcgagctcacggccttcgacaatctcttccg
ccgggtgcccggcgcgctccggggcctcgccaacctgacgcacgctcatt
tcgagcgcagccgcatcgaggccgtggcctccggctcgctgctgggcatg
cggcgtctgcgctcgctcagcctgcaggccaaccgcgtgcgcgcggtgca
tgccggggcctttggcgactgcggcgccctggaggacctgctgctcaacg
acaacctgctggccacgctgcccgccgccgccttccgcggccttcgccgc
ctgcgcaccctcaacctgggcggcaacgcgctgggcagcgtggcacgcgc
ctggttctcagacctggcagagctcgagctgctttacctggaccgcaaca
gcatcacctttgttgaggaaggcgccttccagaacctctcgggcctcctg
gccctgcatctcaatggcaaccgtctcactgtgctctcctgggccgcttt
ccagccaggtttcttcctgggccgcctcttccttttccgcaatccttggc
gctgtgactgccaactggagtggctgcgtgattggatggagggctctggg
cgtgtggctgatgtggcgtgcgcctccccaggctctgtggccggccagga
cctcagccaggtggtctttgagcgctcctctgatggcctctgtgtggacc
ctgatgaactgaactttaccacgtccagtcctggcccgagtccggagcca
gtggccaccactgtgagcaggttcagcagcctcctctccaagctgctggc
cccaagggcccctgtggaggaggtagccaataccacctgggagctggtca
acgtctcgttgaatgacagctttcggtcccatgcagtgatggtcttctgc
tacaaggccacgtttctcttcacctcttgcgtcttgctcagcctggccca
gtatgtggtggtgggcctgcagagggagtga

The rAAV vectors of the present disclosure may be comprised within a virion having a serotype that is selected from the group consisting of AAV serotype 1, AAV serotype 2, AAV serotype 3, AAV serotype 4, AAV serotype 5, AAV serotype 6, AAV serotype 7, AAV serotype 8, AAV serotype 9, or AAV serotype 10, or any other serotype as known to one of ordinary skill in the viral arts.

In related embodiments, the disclosure further provides populations and pluralities of rAAV vectors, virions, infectious viral particles, or host cells that include one or more nucleic acid segments that encode an AAV vector comprising a multi-mutated VP3 protein that includes an ON bipolar cell-specific promoter operably linked to a selected polynucleotide enclosed a therapeutic agent.

The disclosure further provides composition and formulations that include one or more of the proteins, nucleic acid segments, viral vectors, host cells, or viral particles of the present disclosure together with one or more pharmaceutically-acceptable buffers, diluents, or excipients. Such compositions may be included in one or more diagnostic or therapeutic kits, for diagnosing, preventing, treating or ameliorating one or more symptoms of a mammalian disease, injury, disorder, trauma or dysfunction, and in particular, for delivery of a therapeutic agent to ON bipolar cells of the mammalian retina.

The disclosure further includes a method for providing a mammal in need thereof with a diagnostically- or therapeutically-effective amount of a selected therapeutic agent, the method comprising providing to a cell, tissue or organ of a mammal in need thereof, an amount of one or more of the disclosed rAAV multi-capsid mutant vectors; and for a time effective to provide the mammal with a diagnostically- or a therapeutically-effective amount of the selected therapeutic agent.

The disclosure further provides a method for diagnosing, preventing, treating, or ameliorating at least one or more symptoms of a disease, a disorder, a dysfunction, an injury, an abnormal condition, or trauma in a mammal. In an overall and general sense, the method includes at least the step of administering to a mammal in need thereof one or more of the disclosed rAAV vectors, in an amount and for a time sufficient to diagnose, prevent, treat or ameliorate the one or more symptoms of the disease, disorder, dysfunction, injury, abnormal condition, or trauma in the mammal.

The disclosure also provides a method of transducing a population of mammalian cells. In an overall and general sense, the method includes at least the step of introducing into one or more cells of the population, a composition that comprises an effective amount of one or more of the rAAV vectors disclosed herein.

In a further embodiment, the disclosure also provides isolated nucleic acid segments that encode one or more of the AAV mutant capsid proteins as described herein, and provides recombinant vectors, virus particles, infectious virions, and isolated host cells that comprise one or more of the improved rAAV vectors described herein.

Additionally, the present disclosure provides compositions, as well as therapeutic and/or diagnostic kits that include one or more of the disclosed AAV vector compositions, formulated with one or more additional ingredients, or prepared with one or more instructions for their use.

The disclosure also demonstrates methods for making, as well as methods of using the disclosed improved rAAV capsid-mutated vectors in a variety of ways, including, for example, ex situ, in vitro and in vivo applications, methodologies, diagnostic procedures, and/or gene therapy treatment methods. Because many of the improved vectors are resistant to proteasomal degradation, they possess significantly increased transduction efficiencies in vivo making them particularly suited for viral vector-based human gene therapy regimens, and for delivering one or more genetic constructs to selected mammalian cells in vivo and/or in vitro.

In one aspect, the disclosure provides compositions comprising recombinant adeno-associated viral (AAV) vectors, virions, viral particles, and pharmaceutical formulations thereof, useful in methods for delivering genetic material encoding one or more beneficial or therapeutic product(s) to mammalian cells and tissues. In particular, the compositions and methods of the disclosure provide a significant advancement in the art through their use in the treatment, prevention, and/or amelioration of symptoms of one or more mammalian diseases. It is contemplated that human gene therapy will particularly benefit from the present teachings by providing new and improved viral vector constructs for use in the treatment of a number of diverse diseases, disorders, and dysfunctions.

In another aspect, the disclosure concerns modified rAAV vector that encode one or more mammalian therapeutic agents for the prevention, treatment, and/or amelioration of one or more disorders in the mammal into which the vector construct is delivered.

In particular, the disclosure provides rAAV-based expression constructs that encode one or more mammalian therapeutic agent(s) (including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a mammalian disease, dysfunction, injury, and/or disorder.

In one embodiment, the disclosure provides an rAAV vector that comprises at least a first capsid protein comprising at least a first amino acid substitution to a non-native amino acid at one or more surface exposed amino acid residues in an rAAV capid protein, and wherein the vector further additionally includes at least a first nucleic acid segment that encodes at least a first diagnostic or therapeutic agent operably linked to an ON bipolar cell-specific promoter capable of expressing the segment in one or more retinal neurons that has been transformed with the vector.

The surface-exposed amino acid-modified rAAV vectors of the present disclosure may optionally further include one or more enhancer sequences that are each operably linked to the nucleic acid segment. Exemplary enhancer sequences include, but are not limited to, one or more selected from the group consisting of a CMV enhancer, a synthetic enhancer, a bipolar-specific enhancer, a liver-specific enhancer, an vascular-specific enhancer, a brain-specific enhancer, a neural cell-specific enhancer, a lung-specific enhancer, a muscle-specific enhancer, a kidney-specific enhancer, a pancreas-specific enhancer, and an islet cell-specific enhancer.

Exemplary promoters useful in the practice of the disclosure include, without limitation, one or more tissue-specific promoters, including, for example, but not limited to, an ON bipolar cell-specific promoters. The first nucleic acid segment may also further include one or more post-transcriptional regulatory sequences or one or more polyadenylation signals, including, for example, but not limited to, a woodchuck hepatitis virus post-transcription regulatory element, a polyadenylation signal sequence, intron/exon junctions/splicing signals from nyctalopin (nyx), metabotropic glutamate receptor 6-mGluR6 (Grm6), transient receptor potential melastatin 1 (TRPM1), G protein coupled receptor 179 (GPR179), and G proteins, Gβ5, Gβ3, Gα0_{1/2}, Gγ13, RGS7, RGS11 or R9AP, or any combination thereof.

Exemplary diagnostic or therapeutic agents deliverable to host cells by the present vector constructs include, but are not limited to, an agent selected from the group consisting of a polypeptide, a peptide, an antibody, an antigen binding fragment, a ribozyme, a peptide nucleic acid, a siRNA, an RNAi, an antisense oligonucleotide, an antisense polynucleotide, and any combination thereof.

In exemplary embodiments, the improved rAAV vectors of the disclosure will preferably encode at least one diagnostic or therapeutic protein or polypeptide selected from the group consisting of a molecular marker, photosensitive opsins, including, without limitation, rhodopsin, melanopsin, cone opsins, channel rhodopsins, halorhodopsins, bacterial or archea-associated opsins, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, a tumor suppressor, and any combination thereof.

In certain applications, the capsid-modified rAAV vectors of the present disclosure may include one or more nucleic acid segments that encode a therapeutic agent which is a polypeptide selected from the group consisting of nyctalopin (nyx), metabotropic glutamate receptor 6-mGluR6 (Grm6), transient receptor potential melastatin 1 (TRPM1), G protein coupled receptor 179 (GPR179), and G proteins, Gβ5, Gβ3, Gα0_{1/2}, Gγ13, RGS7, RGS11 or R9AP, and any combination thereof. In some embodiments, the polypeptide is a Nyx polypeptide. In some embodiments, the Nyx polypeptide comprises or consists of an amino acid sequence that is encoded by the nucleotide sequence of SEQ ID NO: 13 or SEQ ID NO: 14.

In some embodiments, the disclosure concerns genetically-modified inproved transduction-efficiency rAAV vectors that include at least a first nucleic acid segment that encodes one or more therapeutic agents that alter, inhibit, reduce, prevent, eliminate, or impair the activity of one or more endogenous biological processes in the cell. In particular embodiments, such therapeutic agents may be those that selectively inhibit or reduce the effects of one or more metabolic processes, dysfunctions, disorders, or diseases. In certain embodiments, the defect may be caused by injury or trauma to the mammal for which treatment is desired. In some embodiments, the defect may be caused the over-expression of an endogenous biological compound, while in other embodiments still; the defect may be caused by the under-expression or even lack of one or more endogenous biological compounds.

The genetically-modified rAAV vectors and expression systems of the present disclosure may also further include a second nucleic acid segment that comprises, consists essentially of, or consists of, one or more enhancers, one or more regulatory elements, one or more transcriptional elements, or any combination thereof, that alter, improve, regulate, and/or affect the transcription of the nucleotide sequence of interest expressed by the modified rAAV vectors.

For example, the rAAV vectors of the present disclosure may further include a second nucleic acid segment that comprises, consists essentially of, or consists of, a CMV enhancer, a synthetic enhancer, a cell-specific enhancer, a tissue-specific enhancer, or any combination thereof. The second nucleic acid segment may also further comprise, consist essentially of, or consist of, one or more intron sequences, one or more post-transcriptional regulatory elements, enhancers from nyctalopin (nyx), metabotropic glutamate receptor 6-mGluR6 (Grm6), transient receptor potential melastatin 1 (TRPM1), G protein coupled receptor 179 (GPR179), G proteins, Gβ5, Gβ3, Gα0_{1/2}, Gγ13, RGS7, RGS11 or R9AP, or any combination thereof.

The improved vectors and expression systems of the present disclosure may also optionally further include a polynucleotide that comprises, consists essentially of, or consists of, one or more polylinkers, restriction sites, and/or multiple cloning region(s) to facilitate insertion (cloning) of one or more selected genetic elements, genes of interest, or therapeutic or diagnostic constructs into the rAAV vector at a selected site within the vector.

In further aspects of the present disclosure, the exogenous polynucleotide(s) that may be delivered into suitable host cells by the improved, capsid-modified, rAAV vectors disclosed herein are preferably of mammalian origin, with polynucleotides encoding one or more polypeptides or peptides of human, non-human primate, porcine, bovine, ovine, feline, canine, equine, epine, caprine, or lupine origin being particularly preferred.

The exogenous polynucleotide(s) that may be delivered into host cells by the disclosed capsid-modified viral vectors may, in certain embodiments, encode one or more proteins, one or more polypeptides, one or more peptides, one or more enzymes, or one or more antibodies (or antigen-binding fragments thereof), or alternatively, may express one or more siRNAs, ribozymes, antisense oligonucleotides, PNA molecules, or any combination thereof. When combinational gene therapies are desired, two or more different molecules may be produced from a single rAAV expression system, or alternatively, a selected host cell may be transfected with two or more unique rAAV expression systems, each of which may comprise one or more distinct polynucleotides that encode a therapeutic agent.

In some embodiments, the disclosure also provides capsid-modified rAAV vectors that are comprised within an infectious adeno-associated viral particle or a virion, as well as pluralities of such virions or infectious particles. In some embodiments, the disclosure also provides rAAV vectors that are comprised within an infectious adeno-associated viral particle or a virion that is a capsid-modified AAV particle or virion, as well as pluralities of such virions or infectious particles. Such vectors and virions may be comprised within one or more diluents, buffers, physiological solutions or pharmaceutical vehicles, or formulated for administration to a mammal in one or more diagnostic, therapeutic, and/or prophylactic regimens. The vectors, virus particles, virions, and pluralities thereof of the present disclosure may also be provided in excipient formulations that are acceptable for veterinary administration to selected livestock, exotics, domesticated animals, and companion animals (including pets and such like), as well as to non-human primates, zoological or otherwise captive specimens, and such like.

The disclosure also concerns host cells that comprise at least one of the disclosed capsid protein-modified rAAV expression vectors, or one or more virus particles or virions that comprise such an expression vector. Such host cells are particularly mammalian host cells, with human retinal cells being particularly highly preferred, and may be either isolated, in cell or tissue culture. In the case of genetically modified animal models, the transformed host cells may even be comprised within the body of a non-human animal itself.

In certain embodiments, the creation of recombinant non-human host cells, and/or isolated recombinant human host cells that comprise one or more of the disclosed rAAV vectors is also contemplated to be useful for a variety of diagnostic, and laboratory protocols, including, for example, means for the production of large-scale quantities of the rAAV vectors described herein. Such virus production methods are particularly contemplated to be an improvement over existing methodologies including in particular, those that require very high titers of the viral stocks in order to be useful as a gene therapy tool. The inventors contemplate that one very significant advantage of the present methods will be the ability to utilize lower titers of viral particles in mammalian transduction protocols, yet still retain transfection rates at a suitable level.

Compositions comprising one or more of the disclosed capsid-modified, improved transduction-efficiency rAAV vectors, expression systems, infectious AAV particles, or host cells also form part of the present disclosure, and particularly those compositions that further comprise at least a first pharmaceutically-acceptable excipient for use in therapy, and for use in the manufacture of medicaments for the treatment of one or more mammalian diseases, disorders, dysfunctions, or trauma. Such pharmaceutical compositions may optionally further comprise one or more diluents, buffers, liposomes, a lipid, a lipid complex. Alternatively, the surface exposed amino acid-substituted rAAV vectors of the present disclosure may be contained within or mixed with a plurality of microspheres, nanoparticles, liposomes, or any combination thereof. Pharmaceutical formulations suitable for intravitreal administration to one or both eyes of a human or other mammal are particularly preferred, however, the compositions disclosed herein may also find utility in administration to discreet areas of the mammalian body, including for example, formulations that are suitable for direct injection into one or more organs, tissues, or cell types in the body.

Other aspects of the disclosure concern recombinant adeno-associated virus virion particles, compositions, and host cells that comprise, consist essentially of, or consist of, one or more of the capsid-modified, improved transduction efficiency, rAAV vectors disclosed herein, such as for example pharmaceutical formulations of the vectors intended for intravitreal administration to a mammalian eye.

Kits comprising one or more of the disclosed capsid-modified rAAV vectors (as well as one or more virions, viral particles, transformed host cells or pharmaceutical compositions comprising such vectors); and instructions for using such kits in one or more therapeutic, diagnostic, and/or prophylactic clinical embodiments are also provided by the present disclosure. Such kits may further comprise one or more reagents, restriction enzymes, peptides, therapeutics, pharmaceutical compounds, or means for delivery of the composition(s) to host cells, or to an animal (e.g., syringes, injectables, and the like). Exemplary kits include those for treating, preventing, or ameliorating the symptoms of a disease, deficiency, dysfunction, and/or injury, or may include components for the large-scale production of the viral vectors themselves, such as for commercial sale, or for use by others, including e.g., virologists, medical professionals, and the like.

Another important aspect of the present disclosure concerns methods of use of the disclosed rAAV vectors, virions, expression systems, compositions, and host cells described herein in the preparation of medicaments for diagnosing, preventing, treating or ameliorating at least one or more symptoms of a disease, a dysfunction, a disorder, an abnormal condition, a deficiency, injury, or trauma in an animal, and in particular, in the eye of a vertebrate mammal. Such methods generally involve administration to the eye (e.g., direct administration to the vitreous of one or both eyes) of a mammal in need thereof, one or more of the disclosed vectors, virions, viral particles, host cells, compositions, or pluralities thereof, in an amount and for a time sufficient to diagnose, prevent, treat, or lessen one or more symptoms of such a disease, dysfunction, disorder, abnormal condition, deficiency, injury, or trauma in one or both eyes of the affected animal. The methods may also encompass prophylactic treatment of animals suspected of having such conditions, or administration of such compositions to those animals at risk for developing such conditions either following diagnosis, or prior to the onset of symptoms.

As described above, the exogenous polynucleotide will preferably encode one or more therapeutic proteins, polypeptides, peptides, ribozymes, or antisense oligonucleotides, or a combination of these. In fact, the exogenous polynucleotide may encode two or more such molecules, or a plurality of such molecules as may be desired. When combinational gene therapies are desired, two or more different molecules may be produced from a single rAAV expression system, or alternatively, a selected host cell may be transfected with two or more unique rAAV expression systems, each of which will provide unique heterologous polynucleotides encoding at least two different such molecules.

Compositions comprising one or more of the disclosed rAAV vectors, expression systems, infectious AAV particles, host cells also form part of the present disclosure, and particularly those compositions that further comprise at least a first pharmaceutically-acceptable excipient for use in the manufacture of medicaments and methods involving therapeutic administration of such rAAV vectors. Pharmaceutical formulations suitable for intravitreal administration into one or both eyes of a human or other mammal are particularly preferred.

Another important aspect of the present disclosure concerns methods of use of the disclosed vectors, virions, expression systems, compositions, and host cells described herein in the preparation of medicaments for treating or ameliorating the symptoms of various deficiencies in an eye of a mammal, and in particular one or more deficiencies in human ON bipolar cells. Such methods generally involve intravitreal administration to one or both eyes of a subject in need thereof, one or more of the disclosed vectors, virions, host cells, or compositions, in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms.

Another aspect of the disclosure relates to use of an rAAV vector as described herein to transduce one or more retinal cells (e.g., photoreceptors, ON bipolar cells, and the like). In some embodiments, the one or more retinal cells are ON bipolar cells.

### BRIEF DESCRIPTION OF THE DRAWINGS

For promoting an understanding of the principles of the disclosure, reference will now be made to the embodiments, or examples, illustrated in the drawings and specific language will be used to describe the same. It will, nevertheless be understood that no limitation of the scope of the disclosure is thereby intended. Any alterations and further modifications in the described embodiments, and any further applications of the principles of the disclosure as described herein are contemplated as would normally occur to one of ordinary skill in the art to which the disclosure relates.

The following drawings form part of the present specification and are included to demonstrate certain aspects of the present disclosure. The disclosure may be better understood by reference to the following description taken in conjunction with the accompanying drawings, in which like reference numerals identify like elements, and in which:

FIG. 1 shows the transduction efficiency of unmodified and capsid mutated vectors in vitro. 661W cells were infected with scAAV2, scAAV2(quadY-F), scAAV2(quadY-F+T-V), scAAVS, scAAV5(singleY-F), and scAAVS(doubleY-F) at a multiplicity of infection (MOI) of 10,000. mCherry expression is shown in arbitrary units on the ‘y’ axis, calculated by multiplying the percentage of positive cells by the mean fluorescence intensity in each sample;

FIG. 2A, FIG. 2B, FIG. 2C, FIG. 2D, FIG. 2E, and FIG. 2F show the qualitative comparison of unmodified and capsid mutated AAV vectors in vivo. Fundoscopy (red channel only) of Rho-GFP mice 4 weeks post-injection with unmodified and capsid-mutated scAAV-smCBA-mCherry vectors (1.5×10⁹ vg delivered). Exposure and gain settings were the same across all images; FIG. 2A, FIG. 2B and FIG. 2C show relative mCherry expression in retinas of live mice injected with either unmodified scAAV2 (FIG. 2A), scAAV2 containing 4 typrosine-phenylalanine (Y-F) mutations on its capsid surface (FIG. 2B) and scAAV2 containing 4 Y-F mutations in addition to one threonine-valine (T-V) mutation on its capsid surface (FIG. 2C). mCherry expression was enhanced with addition of capsid mutations (FIG. 2A, FIG. 2B, and FIG. 2C), with scAAV2(quadY-F+T-V)-cmCBA-mCherry exhibiting the highest qualitative levels of mCherry expression (FIG. 2C). scAAVS-based vectors were similarly compared in FIG. 2D, FIG. 2E and FIG. 2F. Neither scAAVS or scAAVS containing a single Y-F mutation conferred any appreciable mCherry expression to retinas of intravitreally injected mice, as assessed by fundoscopy (FIG. 2D and FIG. 2E). scAAV5(doubleY-F)-smCBA-mCherry resulted in modest mCherry expression only in peripapillary retina and retina proximal to blood vessels (FIG. 2F);

FIG. 3A, FIG. 3B and FIG. 3C show the quantitative comparison of unmodified and capsid mutated AAV vectors in vivo. Transduction efficiency of unmodified and capsid-mutated scAAV2 and scAAVS vectors in Rho-GFP mice. FACS analysis was used to quantify the percentage of cells that were GFP positive (PRs), mCherry positive (any retinal cells transduced with AAV) and both GFP and mCherry positive (PRs transduced by AAV). Representative plots for a negative control (uninjected retina) and 2 pooled retinas injected with scAAV2(quadY-F+T-V) are shown in FIG. 3A and FIG. 3B, respectively. Cells that were both GFP and mCherry positive are shown in the top right of FIG. 3A and FIG. 3B and represent the percent of transduced PRs. The bottom right of FIG. 3A and FIG. 3B show cells that were mCherry positive but GFP negative, representing off-target transduction. The percentage of mCherry positive PRs (a measure of in vivo PR transduction efficiency for each vector) in retinas injected with unmodified or capsid-mutated scAAV vectors is shown in FIG. 3C;

FIG. 4A, FIG. 4B, FIG. 4C, FIG. 4D, FIG. 4E, and FIG. 4F show the in vivo analysis of AAV2-based vectors containing the hGRK1 promoter. Fundus images paired with immunohistochemistry of frozen retinal cross-sections from C57BL/6 mice taken 4-weeks' post-injection with AAV2, AAV2(quad Y-F), and AAV2(quad Y-F +T-V) vectors containing hGRK1-GFP (7.5×10⁹ vg delivered). Identical gain and exposures were used for fundoscopy. All tissue sections were imaged at 20×, with identical gain and exposure settings. GFP expression is shown in green. Nuclei were counterstained with DAPI (blue). RPE—retinal pigment epithelium, IS/OS—inner segments/outer segments, ONL—outer nuclear layer, INL—inner nuclear layer, GCL—ganglion cell layer; AAV2-hGRK1-mediated GFP expression is restricted to ganglion cells of intravitreally injected mice (FIG. 4B). AAV2(quadY-F)-hGRK1-mediated GFP expression, albeit modest, is found in ganglion cells and photoreceptors of intravitreally inected mice (FIG. 4D). Of all vectors tested, AAV2(quadY-F+T-V) mediates the most robust GFP expression throughout inner, middle and outer retinal layers including ganglion cells, bipolar cells and photoreceptors (FIG. 4F);

FIG. 5A, FIG. 5B, FIG. 5C, FIG. 5D, FIG. 5E, and FIG. 5F show the in vivo analysis of AAVS-based vectors containing the hGRK1 promoter. Fundus images paired with IHC of frozen retinal cross-sections from C57BL/6 mice taken 4-weeks' post-injection with capsid-mutated AAVS vectors containing hGRK1-GFP. For analysis of AAVS(singleY-F) and AAVS(doubleY-F) vectors 8.5×10¹⁰ vg and 5.3×10⁹ vg were delivered, respectively. Retinal tissue sections containing optic nerve head (FIG. 5B and FIG. 5E) and peripheral retinal cross sections (FIG. 5C and FIG. 5F) are shown. White arrows demarcate the optic nerve head. Identical gain and exposures were used for fundoscopy. All cross sections were imaged at 20×, with identical gain and exposure settings. GFP expression is shown in green. Nuclei were counterstained with DAPI (blue). RPE—retinal pigment epithelium, IS/OS—inner segments/outer segments, ONL—outer nuclear layer, INL—inner nuclear layer, GCL—ganglion cell layer;

FIG. 6A, FIG. 6B, FIG. 6C, and FIG. 6D show the microRNA-mediated regulation of transgene expression. Both hGRK1-GFP and hGRK1-GFP-miR181c were packaged in AAV2(quadY-F+T-V) and delivered intravitreally to C57BL/6 mice (1.5×10¹⁰ vg). Fundoscopy at 4-weeks' post-injection is shown adjacent to immunohistochemistry of frozen retinal cross-sections. Identical gain and exposures were used for fundoscopy. All cross sections were imaged at 20×, with identical gain and exposure settings. GFP expression is shown in green. Nuclei were counterstained with DAPI (blue). RPE—retinal pigment epithelium, IS/OS—inner segments/outer segments, ONL—outer nuclear layer, INL—inner nuclear layer, GCL—ganglion cell layer;

FIG. 7A, FIG. 7B, and FIG. 7C show transduction efficiency of scAAV2(quadY-F) and scAAV2(quadY-F+T-V) in PRs of Rho-GFP mice 1-week post-intravitreal injection;

FIG. 8 shows representative image of a retinal tissue section from a C57BL/6 mouse injected with AAV2(quadY-F+T-V) (5.0×10⁹ vg delivered), stained for GFP and counterstained with DAPI. Merged images are presented at 10× to visualize the full retina;

FIG. 9A, FIG. 9B, FIG. 9C, FIG. 9D, FIG. 9E, FIG. 9F, FIG. 9G, FIG. 9H, and FIG. 9I show in vivo, qualitative analysis of AAV2-based vectors containing the ubiquitous, CBA promoter. Fundus images paired with immunohistochemistry of frozen retinal cross sections from C57BL/6 mice taken 4 weeks post injection with AAV2(tripleY-F), AAV2(triple Y-F+T-V), AAV2(quadY-F), and AAV2(quad Y-F+T-V) vectors containing ubiquitous promoter CBA driving GFP (1.5×10¹⁰ vg delivered.) Identical gain and exposures were used for fundoscopy. Retinal sections were imaged at 5x for visualization of the entire retina from periphery to periphery (FIG. 9B, FIG. 9E, and FIG. 9H), at 20× for detailed analysis of each retinal cell type (FIG. 9C, FIG. 9F, and FIG. 9I) and at 40× for better resolution of outer the retina (inserts in FIG. 9C, FIG. 9F, and FIG. 9I). All sections were imaged with identical gain and exposure settings. GFP expression is shown in green. Nuclei were counterstained with DAPI (blue);

FIG. 10A, FIG. 10B, FIG. 10C, and FIG. 10D are representative images of GFP-positive photoreceptors from a mouse injected intravitreally with AAV2(quadY-F+T-V)-CBA-GFP. Photoreceptors were distinguished from Muller glia processes by counting GFP-positive cell bodies and outer segments (examples demarcated with white arrows);

FIG. 10E is the composite of the four individual images depicted in FIG. 10A-FIG. 10D;

FIG. 11A, FIG. 11B, FIG. 11C, and FIG. 11D are representative images of GFP-positive photoreceptors from a mouse injected intravitreally with AAV2(quadY-F+T-V)-hGRK1-GFP;

FIG. 11E is the composite of the four individual images depicted in FIG. 11A-FIG. 11D;

FIG. 12 shows semi-quantitative comparison of the number of transduced photoreceptors in eyes intravitreally injected with either AAV2(quadY-F+T-V)-hGRK1-GFP or AAV2(quadY-F+T-V)-CBA-GFP. Photoreceptor transduction was measured as a function of GFP expression in these cells within four representative areas of retinas injected with each vector. All areas analyzed were of equal size based on magnification (40×);

FIG. 13A and FIG. 13B show representative 20× (FIG. 13A) and 40× (FIG. 13B) images of AAV2(quadY-F+T-V)-Ple155-GFP vector injected intravitreally in WT mice; fixed frozen retinal cross-sections were stained with antibodies raised against GFP and PCP2. Vector-mediated GFP expression was restricted to target cells (ON bipolars). ONL—outer nuclear layer, INL—inner nuclear layer, IS/OS—inner segments/outer segments, GC—ganglion cell layer. Green: GFP; Red: PCP2, and Blue: DAPI;

FIG. 14 is a representative image of AAV2(quadY-F+T-V)-Ple155-nyx/YFP vector injected intravitreally in WT mice; fixed, frozen retinal cross-sections were stained with an antibody against YFP and PKCalpha. Vector-mediated nyx expression was restricted to target cells (ON bipolar cells). ONL—outer nuclear layer, INL—inner nuclear layer, IS/OS—inner segments/outer segments, GC—ganglion cell layer. Green: GFP; Red: PKCa, and Blue: DAPI;

FIG. 15 is representative image of AAV2(quadY-F+T-V)-Ple155-nyx/YFP; Vector injected intravitreally in nyx/nob (Nyx^{nob}) mice; fixed, frozen retinal cross-sections stained with an antibody against YFP and PKCalpha. Vector-mediated nyx expression was restricted to target cells (ON bipolar cells);

FIG. 16 is representative image of AAV2(quadY-F+T-V)-Ple155-GFP; Vector injected sub-retinally in dystrophic rd16 mice; fixed, frozen retinal cross-sections stained with an antibody against GFP and PKCalpha. Vector-mediated GFP expression was restricted to target cells (ON bipolar cells). **note that only one layer of nuclei remain in ONL because profound photoreceptor degeneration has already occurred; and

FIG. 17 is representative image of AAV2(quadY-F+T-V)-Ple155-GFP; Vector injected intravitreally in dystrophic rd16 mice; fixed, frozen retinal cross-sections stained with an antibody against GFP and PKCalpha. Vector-mediated GFP expression seen in ON bipolar cells **note that only one layer of nuclei remain in ONL because profound photoreceptor degeneration has already occurred.

FIG. 18A shows a set of electroretinograms (ERGs) recorded from two nyx/nob (Nyx^{nob}) mice, each of which were treated with AAV2(quadY-F+T-V)-Ple155-YFP/nyx in one eye (black tracings, AAV TX) while the other eye (grey tracings, no Tx) was untouched. FIG. 18B is a graph showing the magnitude of the positive component obtained to −2.4 log cd s/m2 stimuli.

FIG. 19 shows a series of photographs of a mouse retina stained with TRPM1 and GFP antibodies in an AAV-treated eye. The top panel shows a low magnification image in one plane of the retinal wholemount. Boxes highlight area which is magnified in the middle panel. The left-most column shows TRPM1 staining, the middle column shows GFP staining, and the right-most column shows a merge of the tRPM1 and the GFP staining.

FIG. 20 shows a series of photographs of a mouse stained with with TRPM1 antibody (left panel), GFP antibody (middle panel), or a merge of the TRPM1 and GFP channels with DAPI staining (right panel).

FIG. 21 shows a series of photographs of a 100 um thick retinal slice used for patching bipolar cells. The left panel shows TRPM 1 staining, the middle panel shows GFP staining, and the right panel shows a merge of the TRPM1, GFP and DAPI staining.

FIG. 22A shows a series of photographs of the first cell recorded from a 100 um thick retinal slice used for patching bipolar cells. The left panel shows TRPM 1 staining in a cell filled with sulphur rhodamine, the middle panel shows GFP staining, and the right panel shows a merge of the TRPM1, GFP and DAPI staining. FIG. 22B shows a trace of a capsaicin response in the cell.

FIG. 23 shows a series of traces of a capsaicin response in the cell from a wild-type mouse (left trace), a Nyx^{nob} mouse retina treated with AAV (middle trace, rescue), and an untreated Nyx^{nob} mouse retina (right trace).

FIG. 24A shows a graph of the response amplitude to capsaicin puff delivered at 200 msec in wild-type mouse eyes, Nyx^{nob} mouse eyes injected with AAV (Nob-AAV), and Nyx^{nob} mouse eyes that were untreated.

FIG. 24B shows a graph of the response amplitude to capsaicin puff delivered for 1 second in wild-type mouse eyes, Nyx^{nob} mouse eyes injected with AAV (Nob-AAV), and Nyx^{nob} mouse eyes that were untreated.

FIG. 25 shows a photograph of a non-human primate eye (animal 1) expressing AAV2(quadY-F+T-V)-delivered GFP (top and bottom, left side) and AAV2(quadY-F+T-V)-delivered mCherry (top and bottom, right side). The images were taken 4 weeks post injection.

FIG. 26 shows a photograph of a non-human primate eye (animal 1) expressing AAV2(quadY-F+T-V)-delivered GFP (left side) and AAV2(quadY-F+T-V)-delivered mCherry (right side). The images were taken 4 weeks post injection.

FIG. 27 shows a photograph of a non-human primate eye expressing (animal 2) AAV2(quadY-F+T-V)-delivered GFP (left side) and AAV2(quadY-F+T-V)-delivered mCherry (right side). The images were taken 4 weeks post injection.

FIG. 28 shows a photograph of a non-human primate eye (animal 2) expressing AAV2(quadY-F+T-V)-delivered mCherry. The image was taken 4 weeks post injection.

FIGS. 29A and B show photographs of a non-human primate eye (animal 1, right eye) expressing AAV2(quadY-F+T-V)-delivered GFP. The images were taken 8 weeks post injection.

FIG. 30 shows a photograph of a non-human primate eye (animal 1, left eye) expressing AAV2(quadY-F+T-V)-delivered GFP. The image was taken 8 weeks post injection.

FIG. 31 shows a photograph of a non-human primate eye (animal 2, right eye) expressing AAV2(quadY-F+T-V)-delivered GFP. The image was taken 8 weeks post injection.

FIG. 32 shows a photograph of a non-human primate eye (animal 1, right eye) expressing AAV2(quadY-F+T-V)-delivered GFP (left image) and expressing AAV2(quadY-F+T-V)-delivered mCherry (right image). The image was taken 10.5 weeks post injection.

### BRIEF DESCRIPTION OF THE SEQUENCES

SEQ ID NO:1 is an exemplary amino acid sequence of the capsid protein of the wild-type adeno-associated virus serotype 1 (AAV1);

SEQ ID NO:2 is an exemplary amino acid sequence of the capsid protein of the wild-type adeno-associated virus serotype 2 (AAV2);

SEQ ID NO:3 is an exemplary amino acid sequence of the capsid protein of the wild-type adeno-associated virus serotype 3 (AAV3);

SEQ ID NO:4 is an exemplary amino acid sequence of the capsid protein of the wild-type adeno-associated virus serotype 4 (AAV4);

SEQ ID NO:5 is an exemplary amino acid sequence of the capsid protein of the wild-type adeno-associated virus serotype 5 (AAVS);

SEQ ID NO:6 is an exemplary amino acid sequence of the capsid protein of the wild-type adeno-associated virus serotype 6 (AAV6);

SEQ ID NO:7 is an exemplary amino acid sequence of the capsid protein of the wild-type adeno-associated virus serotype 7 (AAV7);

SEQ ID NO:8 is an exemplary amino acid sequence of the capsid protein of the wild-type adeno-associated virus serotype 8 (AAV8);

SEQ ID NO:9 is an exemplary amino acid sequence of the capsid protein of the wild-type adeno-associated virus serotype 9 (AAV9);

SEQ ID NO:10 is an exemplary amino acid sequence of the capsid protein of the wild-type adeno-associated virus serotype 10 (AAV10);

SEQ ID NO:11 is an exemplary oligonucleotide primer;

SEQ ID NO: 12 is an exemplary Ple 155 promoter sequence;

SEQ ID NO: 13 is an exemplary human Nyx cDNA sequence; and

SEQ ID NO: 14 is an exemplary mouse Nyx cDNA sequence.

It is to be understood that SEQ ID NOs: 1-10 refer to exemplary VP1 capsid proteins and that VP2 and VP3 capsid proteins are shorter variants of the VP1 capsid protein generally having a truncated N-terminus compared to VP1. For example, VP2 of AAV2 may lack the first 137 amino acids of VP1 and VP3 of AAV2 may lack the first 202 amino acids of VP1.

### DESCRIPTION OF ILLUSTRATIVE EMBODIMENTS

Illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would be a routine undertaking for those of ordinary skill in the art having the benefit of this disclosure.

“AAV2(quadY-F+T-V)” is a pentuple capsid protein mutant that is derived from AAV serotype 2, in which four surface-exposed tyrosine (Y) residues are each mutated to a phenylalanine (F) residue (i.e., “tyrosine-to-phenyalanine” or “Y-F” mutations), and one surface-exposed threonine (T) residue is also mutated to a valine (V) residue (i.e., “threonine-to-valine” or a “T-V” mutation). Specifically, the four mutated tyrosine residues correspond to amino acid positions Tyr272, Tyr 444, Tyr500, and Tyr730 in the AAV2 capsid sequence, and the mutated threonine resides corresponds to amino acid position Thr491 in the AAV2 wild-type sequence.

Mutation of the four YF and one TV residues yields the quintuple mutation, (shorthand designation: “Y272F+Y444F+Y500F+Y730F+T491V). When delivered to the vitreous of a mammalian eye, this multi-capsid mutant vector is capable of transducing all retinal cell types with high efficiency. The inventors have shown that the self-complementary (sc) AAV2(quadY-F+T-V) vector containing the ubiquitous ‘smCBA’ promoter was capable of transducing up to 25% of photoreceptors following intravitreal injection in mouse. In addition to photoreceptor transduction, AAV2(quadY-F+T-V)-mediated transduction of bipolar cells was also observed. It was reasoned that incorporation of a bipolar-specific promoter into this capsid mutant would promote transgene expression exclusively in this cell type. Incorporation of the novel “Ple155” promoter (derived from Purkinje cell protein 2 (PCP2) regulatory region) into the AAV2 quadYF+TV multi capsid mutant vector led to transgene expression exclusively in ON bipolar cells of mice intravitreally injected with this vector.

The unique ability of the disclosed vectors to selectively- and exclusively-target bipolar cells facilitates multiple uses in vivo. First, it aids in the development of gene replacement strategies for inherited retinal diseases associated with mutations in bipolar-specific genes (e.g., congenital stationary night blindness). In addition, and perhaps more importantly, it facilitates the development of new optogenetic therapies for patients that have lost the ability to process light via traditional phototransduction in rod/cone photoreceptors (e.g., either because their photoreceptors have degenerated and/or because they are rendered dysfunctional). Optogenetics is a technique that confers light sensitivity to neurons via expression of a light-sensitive channel. Importantly this technique can now be achieved using the novel AAV capsid mutant vectors disclosed herein.

Patients with extensive retinal degeneration who lack their naturally photosensitive retinal neurons (photoreceptors), or those with dysfunctional photoreceptors may benefit immensely from technology that confers light sensitivity to bipolar cells (secondary neurons immediately downstream from photoreceptors). Such newly light-sensitive bipolar cells would initiate the conversion of light into an electrical signal which would be send downstream to ganglion cells, through the optic nerve and to the brain where it would be interpreted as vision. Imparting light sensitivity to ON bipolar cells is a much more attractive strategy for restoring useful vision to patients, as they are the neurons furthest ‘upstream’ of the processing centers in the brain. Other related attempts to restore light sensitivity (such as those targeting light-sensitive channels to ganglion cells—the cells furthest downstream from photoreceptors in the retina) have resulted in some light perception to patients, but it is likely that the electrical signals would be more refined if the channels were delivered to neurons farther upstream, e.g., by ON bipolar cells. Accordingly, in some embodiments, rAAV particles and vectors described herein can be used to deliver a light-sensitive channel to an ON bipolar cell. In some embodiments, a rAAV vector polynucleotide comprises a sequence that encodes a light-sensitive channel. Exemplary light-sensitive channels include rhodopsin, melanopsin, cone opsins, channel rhodopsins (e.g., channelrhodopsin-2), halorhodopsins, bacterial or archea-associated opsins (e.g., bacteriorhodopsin), and light-gated excitatory mammalian ion channel light-gated ionotropic glutamate receptor (LiGluR, see, e.g., Gaub et al. Restoration of visual function by expression of a light-gated mammalian ion channel in retinal ganglion cells or ON-bipolar cells. Proc Natl Acad Sci USA. 2014 Dec. 23; 111(51):E5574-83.).

Of particular significance, this disclosure provides the first demonstration that specific ON bipolar cell targeting can be achieved with a vector using less-invasive, intravitreal injection. Current vectors capable of transducing middle retinal neurons such as ON bipolars require subretinal delivery, and their clinical use requires full-blown vitreoretinal surgery performed in a well-staffed surgical center. In contrast, delivery of next-generation vectors capable of transducing all layers of the retina from the vitreous would involve nothing more than the standard outpatient procedure required for injection of wet age-related macular degeneration drugs, Lucentis® and Avastin®. Such vectors could be administered in clinic rather than a surgical suite, thereby increasing accessibility of gene therapies to much larger patient populations.

Optogenetic strategy for delivering light sensitive channels specifically to ON bipolar cells in patients that have pronounced photoreceptor degeneration or dysfunction. This would impart light sensitivity to ON bipolars, allowing them to send electrical signals to downstream neurons and eventually to the brain, where they would be processed as vision. Delivery to ON bipolars would allow more refinement than other optogenetic strategies, which aim to make ganglion cells light sensitive. Furthermore, this vector/promoter combination would allow for delivery of said light channels safely, via the vitreous. This is especially important in patients with pronounced photoreceptor degeneration (e.g., retinitis pigmentosa patients), as their retinas are too fragile to withstand the surgical impact of a subretinal injection.

Gene replacement therapies suitable for use in the present disclosure include, without limitation, those for the treatment, or the amelioration of one or more symptoms of, inherited retinal diseases including, for example, those caused by one or more mutations in one or more genes expressed in ON bipolar cells (e.g., congenital stationary night blindness), or one or more diseases associated with degeneration of bipolar cells (for example melanoma-associated retinopathy [MAR]). Other exemplary diseases include retinitis pigmentosa, cone-rod dystrophy, Leber congenital amaurosis, and late stage age-related macular degeneration. Importantly, rAAV vectors disclosed herein may be formulated for delivery directly to the vitreous, which is a far less invasive protocol, than subretinal injection, which is currently the state of the art).

The present disclosure provides the first AAV vectors that include a modified AAV capsid and a promoter that has been shown to specifically target ON bipolar cells via the vitreous. In an exemplary embodiment, an AAV2(quadY-F+T-V)-Ple155 combination vector effectively targeted ON bipolar cells.

The present disclosure provides an additional advantage over existing technologies because 1) it is highly specific for ON bipolar cells, and 2) it can be applied following less-invasive intravitreal injection. The patients most likely to receive bipolar-targeted gene replacement therapy or optogenetic treatment will have pronounced photoreceptor degeneration. As such, their retinas will be relatively fragile and incapable of withstanding the surgical impact of a subretinal injection. The ability to target ON bipolars via the vitreous minimizes risk, simplifies the overall therapeutic process, and increases accessibility. Clinical treatment using the disclosed vectors that are capable of transducing bipolar cells from the vitreous would involve only a standard, outpatient procedure, similar to that currently required for treatment of wet age-related macular degeneration with drugs such as ranibizumab (LUCENTIS®) and bevacizumab (AVASTIN®), both from Genentech USA, Inc., South San Francisco, Calif., USA. The disclosed vector constructs can be administered in clinic, rather than in a surgical suite, thereby increasing accessibility of gene therapies to much larger patient populations.

rAAV Vectors

Recombinant adeno-associated virus (AAV) vectors have been used successfully for in vivo gene transfer in numerous pre-clinical animal models of human disease, and have been used successfully for long-term expression of a wide variety of therapeutic genes (Daya and Berns, 2008; Niemeyer et al., 2009; Owen et al., 2002; Keen-Rhinehart et al., 2005; Scallan et al., 2003; Song et al., 2004). AAV vectors have also generated long-term clinical benefit in humans when targeted to immune-privileged sites, e.g., ocular delivery for Leber's congenital amaurosis (Bainbridge et al., 2008; Maguire et al., 2008; Cideciyan et al., 2008). A major advantage of this vector is its comparatively low immune profile, eliciting only limited inflammatory responses and, in some cases, even directing immune tolerance to transgene products (LoDuca et al., 2009). Nonetheless, the therapeutic efficiency, when targeted to non-immune privileged organs, has been limited in humans due to antibody and CD8⁺ T cell responses against the viral capsid, while in animal models, adaptive responses to the transgene product have also been reported (Manno et al., 2006; Mingozzi et al., 2007; Muruve et al., 2008; Vandenberghe and Wilson, 2007; Mingozzi and High, 2007). These results suggested that immune responses remain a concern for AAV vector-mediated gene transfer.

Adeno-associated virus (AAV) is considered the optimal vector for ocular gene therapy due to its efficiency, persistence and low immunogenicity (Daya and Berns, 2008). Identifying vectors capable of transducing PRs via the vitreous will rely partially on identifying which serotypes have native tropism for this cell type following local delivery. Several serotypes have been used to successfully target transgene to PRs following subretinal injection (including, e.g., AAV2, AAVS and AAV8) with all three demonstrating efficacy in proof of concept experiments across multiple species (e.g., mouse, rat, dog, pig and non-human primate) (Ali et al., 1996; Auricchio et al., 2001; Weber et al., 2003; Yang et al., 2002; Acland et al., 2001; Vandenberghe et al., 2011; Bennett et al., 1999; Allocca et al., 2007; Petersen-Jones et al., 2009; Lotery et al., 2003; Boye et al., 2012; Stieger et al., 2008; Mussolino et al., 2011; Vandenberghe et al., 2011).

Studies comparing their relative efficiency following subretinal delivery in the rodent show that both AAVS and AAV8 transduce PRs more efficiently than AAV2, with AAV8 being the most efficient (Yang et al., 2002; Allocca et al., 2007; Rabinowitz et al., 2002; Boye et al., 2011; Pang et al., 2011). It was previously shown that AAV2 and AAV8 vectors containing point mutations of surface-exposed tyrosine residues (tyrosine to phenylalanine,Y-F) display increased transgene expression in a variety of retinal cell types relative to unmodified vectors following both subretinal and intravitreal injection (Petrs-Silva et al., 2009; Petrs-Silva et al., 2011). Of the vectors tested, an AAV2 triple mutant (designated “triple Y-F”) exhibited the highest transduction efficiency following intravitreal injection whereas an AAV2 quadruple mutant (“quad Y-F”) exhibited the novel property of enhanced transduction of outer retina (Petrs-Silva et al., 2011). In some embodiments, a rAAV vector polynucleotide (e.g., comprising an ON Bipolar cell-specific promoter) is encapsidated in an AAV2, AAV5, or AAV8 capsid. In some embodiments, the rAAV vector polynucleotide is encapsidated in a modified capsid as described herein (e.g., AAV2(quadY-F+T-V)).

Further improvements in transduction efficiency may be achieved via directed mutagenesis of surface-exposed threonine (T) residues to either valine (V) or alanine (A). Both Y-F and T-V/T-A mutations increase efficiency by decreasing phosphorylation of capsid and subsequent ubiquitination as part of the proteosomal degradation pathway (Zhong et al., 2008; Aslanidi et al., In Press; Gabriel et al., 2013). It was found that the transduction profile of intravitreally-delivered AAV is heavily dependent upon the injection procedure itself. Due to the small size of the mouse eye, it is not uncommon for trans-scleral, intravitreal injections to result in damage to the retina that might allow delivery of some vector directly to the subretinal space.

In some embodiments, a rAAV nucleic acid vector or rAAV vector polynucleotide described herein comprises inverted terminal repeat sequences (ITRs), such as those derived from a wild-type AAV genome, such as the AAV2 genome. In some embodiments, the rAAV nucleic acid vector further comprises a transgene (also referred to as a heterologous nucleic acid molecule) operably linked to a promoter and optionally, other regulatory elements, wherein the ITRs flank the transgene. In some embodiments, the promoter is an ON bipolar cell specific promoter. In one embodiment, the transgene encodes a therapeutic agent or diagnostic agent of interest.

Exemplary rAAV nucleic acid vectors or or rAAV vector polynucleotides useful according to the disclosure include single-stranded (ss) or self-complementary (sc) AAV nucleic acid vectors or polynucleotides, such as single-stranded or self-complementary recombinant viral genomes.

Methods of producing rAAV particles and nucleic acid/polynucleotide vectors are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid containing the nucleic acid vector may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids is a first helper plasmid comprising a rep gene and a cap gene and a second helper plasmid comprising a E1a gene, a E1b gene, a E4 gene, a E2a gene, and a VA gene. In some embodiments, the rep gene is a rep gene derived from AAV2 and the cap gene is derived from AAV2 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDPSrs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Calif.; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy,Vol. 7, 839-850; Kronenberg et al. (2005), A Conformational Change in the Adeno-Associated Virus Type 2 Capsid Leads to the Exposure of Hidden VP1 N Termini , Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

Uses for Improved, Capsid-Modified rAAV Vectors

The present disclosure provides compositions including one or more of the disclosed surface exposed amino acid capsid-modified rAAV vectors comprised within a kit for diagnosing, preventing, treating or ameliorating one or more symptoms of a mammalian disease, injury, disorder, trauma or dysfunction. Such kits may be useful in diagnosis, prophylaxis, and/or therapy, and particularly useful in the treatment, prevention, and/or amelioration of one or more defects in the mammalian eye as discussed herein. The disclosure also provides for the use of a composition disclosed herein in the manufacture of a medicament for treating, preventing or ameliorating the symptoms of a disease, disorder, dysfunction, injury or trauma, including, but not limited to, the treatment, prevention, and/or prophylaxis of a disease, disorder or dysfunction, and/or the amelioration of one or more symptoms of such a disease, disorder or dysfunction. Likewise, the disclosure also provides a method for treating or ameliorating the symptoms of such a disease, injury, disorder, or dysfunction in one or both eyes of a mammal, and of a human in particular. Such methods generally involve at least the step of administering to a mammal in need thereof, one or more of the multi-surface exposed amino acid residue substituted, VP3 capid protein-modified rAAV vectors as disclosed herein, in an amount and for a time sufficient to treat or ameliorate the symptoms of such a disease, injury, disorder, or dysfunction in one or both eyes of the mammal.

The disclosure also provides a method for providing to a mammal in need thereof, a therapeutically-effective amount of the rAAV compositions of the present disclosure, in an amount, and for a time effective to provide the patient with a therapeutically-effective amount of the desired therapeutic agent(s) encoded by one or more nucleic acid segments comprised within the rAAV vector. Preferably, the therapeutic agent is selected from the group consisting of a polypeptide, a peptide, an antibody, an antigen-binding fragment, a ribozyme, a peptide nucleic acid, an siRNA, an RNAi, an antisense oligonucleotide, an antisense polynucleotide, a diagnostic marker, a diagnostic molecule, a reporter molecule, and any combination thereof.

Pharmaceutical Compositions Comprising Capsid-Mutated rAAV Vectors

One important aspect of the present methodology is the fact that the improved rAAV vectors described herein permit the delivery of smaller titers of viral particles in order to achieve the same transduction efficiency as that obtained using higher levels of conventional, non-surface capsid modified rAAV vectors. To that end, the amount of AAV compositions and time of administration of such compositions will be within the purview of the skilled artisan having benefit of the present teachings. In fact, the inventors contemplate that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the AAV vector compositions, either over a relatively short, or over a relatively prolonged period, as may be determined by the medical practitioner overseeing the administration of such compositions. For example, the number of infectious particles administered to a mammal may be approximately 10⁷, 10⁸, 10⁹, 10¹⁰, 10¹¹, 10¹², 10¹³, or even higher, infectious particles/mL, given either as a single dose (or divided into two or more administrations, etc.,) as may be required to achieve therapy of the particular disease or disorder being treated. In fact, in certain embodiments, it may be desirable to administer two or more different rAAV vector-based compositions, either alone, or in combination with one or more other diagnostic agents, drugs, bioactives, or such like, to achieve the desired effects of a particular regimen or therapy. In most rAAV-vectored, gene therapy-based regimens, the inventors contemplate that lower titers of infectious particles will be required when using the modified-capsid rAAV vectors described herein, as compared to the use of equivalent wild-type, or corresponding “un-modified” rAAV vectors.

As used herein, the terms “engineered” and “recombinant” cells are intended to refer to a cell into which an exogenous polynucleotide segment (such as DNA segment that leads to the transcription of a biologically active molecule) has been introduced. Therefore, engineered cells are distinguishable from naturally occurring cells, which do not contain a recombinantly introduced exogenous DNA segment. Engineered cells are, therefore, cells that comprise at least one or more heterologous polynucleotide segments introduced through the hand of man.

To express a therapeutic agent in accordance with the present disclosure one may prepare a tyrosine-modified rAAV expression vector that comprises a therapeutic agent-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (i.e., 3′ of) the chosen promoter. The “upstream” promoter stimulates transcription of the DNA and promotes expression of the encoded polypeptide. This is the meaning of “recombinant expression” in this context. Particularly preferred recombinant vector constructs are those that comprise a capsid-protein modified rAAV vector that contains an ON Bipolar cell-specific promoter operably linked to a nucleic acid segment encoding one or more ocular therapeutic agents. Such vectors are described in detail herein.

When the use of such vectors is contemplated for introduction of one or more exogenous proteins, polypeptides, peptides, ribozymes, and/or antisense oligonucleotides, to a particular cell transfected with the vector, one may employ the capsid-modified rAAV vectors disclosed herein to deliver one or more exogenous polynucleotides to a selected host cell, and preferably, to one or more selected cells within the mammalian eye.

The genetic constructs of the present disclosure may be prepared in a variety of compositions, and may also be formulated in appropriate pharmaceutical vehicles for administration to human or animal subjects. The rAAV molecules of the present disclosure and compositions comprising them provide new and useful therapeutics for the treatment, control, and amelioration of symptoms of a variety of disorders, diseases, injury, and/or dysfunctions of the mammalian eye.

Exemplary Definitions

In accordance with the present disclosure, polynucleotides, nucleic acid segments, nucleic acid sequences, and the like, include, but are not limited to, DNAs (including and not limited to genomic or extragenomic DNAs), genes, peptide nucleic acids (PNAs) RNAs (including, but not limited to, rRNAs, mRNAs and tRNAs), nucleosides, and suitable nucleic acid segments either obtained from natural sources, chemically synthesized, modified, or otherwise prepared or synthesized in whole or in part by the hand of man.

Unless defined otherwise, all technical and scientific terms used herein have the same meaning as commonly understood by one of ordinary skill in the art to which this disclosure belongs. Although any methods and compositions similar or equivalent to those described herein can be used in the practice or testing of the present disclosure, the preferred methods and compositions are described herein. For purposes of the present disclosure, the following terms are defined below:

The term “subject,” as used herein, describes an organism, including mammals such as primates, to which treatment with the compositions according to the present disclosure can be provided. Mammalian species that can benefit from the disclosed methods of treatment include, but are not limited to, apes; chimpanzees; orangutans; humans; monkeys; domesticated animals such as dogs and cats; livestock such as horses, cattle, pigs, sheep, goats, and chickens; and other animals such as mice, rats, guinea pigs, and hamsters.

The term “treatment” or any grammatical variation thereof (e.g., treat, treating, and treatment etc.), as used herein, includes but is not limited to, alleviating a symptom of a disease or condition; and/or reducing, suppressing, inhibiting, lessening, ameliorating or affecting the progression, severity, and/or scope of a disease or condition.

The term “effective amount,” as used herein, refers to an amount that is capable of treating or ameliorating a disease or condition or otherwise capable of producing an intended therapeutic effect.

The term “promoter,” as used herein refers to a region or regions of a nucleic acid sequence that regulates transcription.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “vector,” as used herein, refers to a nucleic acid molecule (typically comprised of DNA) capable of replication in a host cell and/or to which another nucleic acid segment can be operatively linked so as to bring about replication of the attached segment. A plasmid, cosmid, or a virus is an exemplary vector. In some embodiments, a vector is packaged with proteins.

The term “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988).

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

### Exemplary Embodiment

Exemplary, non-limiting embodiments are provided below.
Embodiment 1. A recombinant adeno-associated viral (rAAV) vector comprising:

a polynucleotide that encodes a modified capsid protein, wherein the modified capsid protein comprises at least a first non-native amino acid at a position that corresponds to a surface-exposed amino acid residue in the wild-type AAV2 capsid protein, and further wherein the transduction efficiency of a virion comprising the modified capsid protein is higher than that of a virion comprising a corresponding, unmodified wild-type capsid protein, and

wherein the polynucleotide further comprises a nucleic acid segment that encodes a diagnostic or therapeutic molecule operably linked to an ON bipolar cell-specific promoter that is capable of expressing the nucleic acid segment in one or more middle retinal neuron cells of a mammalian eye.
Embodiment 2. The rAAV vector of embodiment 1, wherein the modified capsid protein comprises three or more non-native amino acid substitutions at positions corresponding to three distinct surface-exposed amino acid residues of the wild-type AAV2 capsid protein as set forth in SEQ ID NO:2; or to three distinct surface-exposed amino acid residues corresponding thereto in any one of the wild-type AAV1, AAV3, AAV4, AAV5, AAV6, AAV7, AAV9, or AAV10 capsid proteins, as set forth, respectively, in SEQ ID NO:1, SEQ ID NO:3, SEQ ID NO:4, SEQ ID NO:5, SEQ ID NO:6, SEQ ID NO:7, SEQ ID NO:9, or SEQ ID NO:10, or any combination thereof.
Embodiment 3. The rAAV vector of embodiment 2, wherein the non-native amino acid substitutions occur at amino acid residues:
   (a) Y272, Y444, Y500, and Y730;
   (b) Y272, Y444, Y500, Y700, and Y730;
   (c) Y272, Y444, Y500, Y704, and Y730;
   (d) Y252, Y272, Y444, Y500, Y704, and Y730;
   (e) Y272, Y444, Y500, Y700, Y704, and Y730;
   (f) Y252, Y272, Y444, Y500, Y700, Y704, and Y730;
   (g) Y444, Y500, Y730, and T491;
   (h) Y444, Y500, Y730, and S458;
   (i) Y444, Y500, Y730, S662, and T491;
   (j) Y444, Y500, Y730, T550, and T491; or
   (k) Y444, Y500, Y730, T659, and T491,

of the wild-type AAV2 capsid protein as set forth in SEQ ID NO:2, or at equivalent amino acid positions corresponding thereto in any one of the wild-type AAV1, AAV3, AAV4, AAV5, AAV6, AAV7, AAV9, or AAV10 capsid proteins, as set forth, respectively, in SEQ ID NO:1, SEQ ID NO:3, SEQ ID NO:4, SEQ ID NO:5, SEQ ID NO:6, SEQ ID NO:7, SEQ ID NO:9, or SEQ ID NO:10, or any combination thereof.
Embodiment 4. The rAAV vector of embodiment 3, comprising the amino acid substitutions Y272F, Y444F, Y500F, Y730F, and T491V in a wild-type AAV2 capsid protein, or equivalent amino acid substitutions at the corresponding residues in any one of the wild-type AAV1, AAV3, AAV4, AAV5, AAV6, AAV7, AAV9, or AAV10 capsid proteins.
Embodiment 5. The rAAV vector of embodiment 1, wherein the transduction efficiency of a virion comprising the modified vector is about 2- to about 50-fold higher in the one or more middle retinal neuron cells than that of a virion that comprises a corresponding, unmodified, wild-type capsid protein.
Embodiment 6. The rAAV vector of embodiment 1, wherein the nucleic acid segment further comprises an enhancer, a post-transcriptional regulatory sequence, a polyadenylation signal, or any combination thereof, operably linked to the nucleic acid segment encoding the therapeutic agent.
Embodiment 7. The rAAV vector of embodiment 1, wherein the ON Bipolar cell-specific promoter is obtained from a mammalian purkinje cell protein 2 (PCP2) regulatory region.
Embodiment 8. The rAAV vector of embodiment 1, wherein the nucleic acid segment expresses or encodes in one or more middle retinal neurons of a mammalian eye, a polypeptide, a peptide, a ribozyme, a peptide nucleic acid, an siRNA, an RNAi, an antisense oligonucleotide, an antisense polynucleotide, an antibody, an antigen binding fragment, or any combination thereof.
Embodiment 9. A method for providing a mammal in need thereof with a therapeutically-effective amount of a selected therapeutic agent, the method comprising intravitreally administering to one or both eyes of the mammal, an amount of the rAAV vector of embodiment 1; and for a time effective to provide the mammal with a therapeutically-effective amount of the selected therapeutic agent.
Embodiment 10. A method for treating or ameliorating one or more symptoms of a disease, a disorder, a dysfunction, an injury, an abnormal condition, or trauma in a mammal, the method comprising, intravitreally administering to one or both eyes of the mammal in need thereof, the rAAV vector of embodiment 1, in an amount and for a time sufficient to treat or ameliorate the one or more symptoms of the disease, the disorder, the dysfunction, the injury, the abnormal condition, or the trauma in the mammal.
Embodiment 11. A method for expressing a nucleic acid segment in one or more retinal cells of a mammal, the method comprising: intravitreally administering to one or both eyes of the mammal the rAAV vector of embodiment 1, wherein the polynucleotide further comprises at least a first polynucleotide that comprises a ON bipolar cell-specific promoter operably linked to at least a first nucleic acid segment that encodes a therapeutic agent, for a time effective to produce the therapeutic agent in the one or more retinal cells of the mammal.
Embodiment 12. The method of embodiment 11, wherein the human has, is suspected of having, is at risk for developing, or has been diagnosed with at least a first retinal disorder, a first retinal disease, or a first retinal dystrophy, or any combination thereof.
Embodiment 13. The method of embodiment 12, wherein the retinal disease or disorder is retinitis pigmentosa, melanoma-associated retinopathy, or congenital stationary night blindness.
Embodiment 14. The method of embodiment 11, wherein the human is a neonate, a newborn, an infant, or a juvenile.
Embodiment 15. The method of embodiment 11, wherein production of the therapeutic agent a) preserves one or more ON bipolar cells, b) restores one or more rod- and/or cone-mediated functions, c) restores visual behavior in one or both eyes, or d) any combination thereof.
Embodiment 16. The method of embodiment 11, wherein production of the therapeutic agent persists in the one or more retinal cells substantially for a period of at least three months following a single intravitreal administration of the rAAV vector into the one or both eyes of the mammal.
Embodiment 17. The method of embodiment 16, wherein production of the therapeutic agent persists in the one or more retinal cells substantially for a period of at least six months following a single intravitreal administration.
Embodiment 18. The method of embodiment 11, wherein the vector is a self-complementary rAAV (scAAV).
Embodiment 19. The method of embodiment 11, wherein the polynucleotide further comprises at least a first enhancer or at least a first mammalian intron sequence operably linked to the first nucleic segment.
Embodiment 20. The method of embodiment 11, wherein the vector is provided to the one or both eyes by administration of an infectious adeno-associated viral particle, an rAAV virion, or a plurality of infectious rAAV particles.
Embodiment 21. The method of embodiment 11, wherein the mammal is human.
Embodiment 22. The method of embodiment 11, wherein the therapeutic agent is an agonist, an antagonist, an anti-apoptosis factor, an inhibitor, a receptor, a cytokine, a cytotoxin, an erythropoietic agent, a glycoprotein, a growth factor, a growth factor receptor, a hormone, a hormone receptor, an interferon, an interleukin, an interleukin receptor, a nerve growth factor, a neuroactive peptide, a neuroactive peptide receptor, a protease, a protease inhibitor, a protein decarboxylase, a protein kinase, a protein kinsase inhibitor, an enzyme, a receptor binding protein, a transport protein or an inhibitor thereof, a serotonin receptor, or an uptake inhibitor thereof, a serpin, a serpin receptor, a tumor suppressor, a chemotherapeutic, or any combination thereof.

### EXAMPLES

The following examples are included to demonstrate certain embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### EXAMPLE 1

### Highly-Selective Transduction of ON Bipolar Cells via Intravitreal Delivery Using Capsid-Mutated AAV Vectors

In this example, novel compositions and methods are provided for quantifying transduction efficiency in vivo using knock-in mice bearing a human rhodopsin-enhanced green fluorescent protein (EGFP) fusion gene (RhoGFP mice) (Wensel et al., 2005), AAV vectors driving mCherry, and subsequent fluorescent activated cell sorting (FACS) to quantify both ‘on-target’ PR transduction (GFP and mCherry positive cell population) and ‘off-target’ retinal cell types (GFP negative, mCherry positive cell population). This method for scoring intravitreally-delivered, AAV-mediated PR transduction can be applied toward development of additional vectors intended for the treatment of inherited retinal disease.

With the enhanced serotypes identified, a reduction in off-target transgene expression was achieved by incorporating the human rhodopsin kinase (hGRK1) promoter in vectors. hGRK1 has demonstrated PR exclusive transduction when incorporated into AAV vectors delivered subretinally to mice and non-human primates (Boye et al., 2012; Khani et al., 2007). Similar to methods previously described (Karali et al., 2011), transgene expression was further restricted to PRs by incorporating multiple target sequences for miR181, an miRNA endogenously expressed in cells of the inner and middle retina.

Surface-exposed tyrosine (Y) and threonine (T) residues on the capsids of AAV2 and AAV5 were changed to phenylalanine (F) and valine (V), respectively. Transduction efficiencies of self-complimentary, capsid-mutant and unmodified AAV vectors containing the smCBA promoter and mCherry cDNA were initially scored in vitro using a cone photoreceptor cell line. Capsid mutants exhibiting the highest transduction efficiencies relative to unmodified vectors were then injected intravitreally into transgenic mice constitutively expressing a Rhodopsin-GFP fusion protein in rod photoreceptors (Rho-GFP mice). Photoreceptor transduction was quantified by fluorescent activated cell sorting (FACS) by counting cells positive for both GFP and mCherry. To explore the utility of the capsid mutants, standard, (non-self-complementary) AAV vectors containing the human rhodopsin kinase promoter (hGRK1) were made. Vectors were intravitreally injected in wildtype mice to assess whether efficient expression exclusive to photoreceptors was achievable. To restrict off-target expression in cells of the inner and middle retina, subsequent vectors incorporated multiple target sequences for miR181, an miRNA endogenously expressed in the inner and middle retina. Results showed that AAV2 containing four Y to F mutations combined with a single T to V mutation (quadY-F+T-V) transduced photoreceptors most efficiently. Robust photoreceptor expression was mediated by AAV2(quadY-F+T-V)-hGRK1-GFP. Observed off-target expression was reduced by incorporating target sequence for a miRNA highly expressed in inner/middle retina, miR181c.

Methods

Vector Production: The following vector plasmid constructs were cloned and packaged in unmodified AAV serotypes 2 and 5, and capsid mutant derivatives of these serotypes; self-complementary small chicken β-actin driving mCherry (sc-smCBA-mCherry), standard (non self-complimentary) human rhodop sin kinase driving green fluorescent protein (hGRK1-GFP), and standard full length chicken β-actin driving GFP (CBA-GFP). Promoter constructs were identical to those previously described (Khani et al., 2007; Haire et al., 2006; Burger et al., 2004). A hGRK1-GFP-miR181c construct was also generated and packaged in AAV2(quad Y-F+T-V) by inserting four tandem copies of complementary sequence for mature miR-181 (5′-ACTCACCGACAGGTTGAA-3′) (SEQ ID NO:11) (“Atlas of miRNA distribution,” http://mirneye.tigem.it/) immediately downstream of GFP, similar to previous reports (Karali et al., 2011).

AAV2 and AAV5 capsid mutants were generated by directed mutagenesis of surface-exposed tyrosine and threonine residues with the QuickChange™ Multi Site-Directed Mutagenesis Kit (Agilent Technologies, Santa Clara, Calif., USA). Selected tyrosine residues were mutated to phenylalanine (Y-F) whereas threonine residues were mutated to valine (T-V) (Zhong et al., 2008). Table 1 describes amino acid location of mutations for experimental mutant vectors. All vectors were packaged, purified, and titered according to previously described methods (Jacobson et al., 2006; Zolotukhin et al., 2002).

TABLE 1
NOMENCLATURE FOR CAPSID-MUTATED
VECTORS WITH DESCRIPTION OF AMINO
ACID LOCATION OF MUTATION

| Vector Nomenclature | Mutation |
|---|---|
| AAV2(tripleY-F) | Y272F + Y444F + Y730F |
| AAV2(tripleY-F + T-V) | Y272F + Y444F + Y730F + T491V |
| AAV2(quadY-F) | Y272F + Y444F + Y500F + Y730F |
| AAV2(quadY-F + T-V) | Y272F + Y444F + Y500F + Y730F + T491V |
| AAV5(singleY-F) | Y719F |
| AAV5(doubleY-F) | Y263F + Y719F |

Cell lines: 661W cone cells (Tan et al., 2004) (University of Oklahoma Health Sciences Center, Oklahoma City, Okla., USA) were passaged by dissociation in 0.05% (wt./vol.) trypsin and 0.02% (wt./vol.) EDTA, followed by re-plating at a split ratio ranging from 1:3 to 1:5 in T75 flasks. Cells were maintained in DMEM containing 10% FBS, 300 mg/L glutamine, 23 mg/L putrescine, 40 μL of β-mercaptoethanol, and 40 μg of hydrocortisone-21-hemisuccinate and progesterone. The media also contained penicillin (90 U/mL) and streptomycin (0.09 mg/mL). Cultures were incubated at 37° C. (Al-Ubaidi et al., 2008).

Infections and FACS analysis: 661W cells were plated in 96-well plates at a concentration of 1.0×10⁴ cells/well. The following day, cells were infected at 10,000 p/cell with sc-smCBA-mCherry packaged in unmodified and modified AAV2 or AAV5 vectors. Three days post-infection, fluorescent microscopy at a fixed exposure was performed, cells were detached and FACS analysis was used to quantify reporter protein (mCherry) fluorescence. Transduction efficiency (mCherry expression) of each AAV vector was calculated as previously reported by multiplying the percentage of positive cells by the mean fluorescence intensity in each sample (Boye et al., 2011).

Animals: Vectors were injected in 1-month-old C57BL/6 mice (The Jackson Laboratory, Bar Harbor, Me., USA) and in 1-month-old heterozygote Rho-GFP mice, knock-in mice bearing human rhodopsin-GFP fusion gene (University of Alabama-Birmingham).

Ethics: All mice were maintained and handled in animal care facilities in accordance with the ARVO statement for Use of Animals in Ophthalmic and Vision Research and the guidelines of the Institutional Animal Care and Use Committee of the University of Florida. Animal work performed in this study was approved by UF's IACUC (animal protocol #201207573).

Intravitreal injections: Prior to vector administration, mice were anesthetized with ketamine (72 mg/kg)/xylazine (4 mg/kg) by intraperitoneal injection. Eyes were dilated with 1% atropine and 2.5% phenylephrine. 1.5 μL of unmodified or capsid-mutated vector was delivered to the intravitreal cavity of adult mice. An aperture was made 0.5 mm posterior to the limbus with a 32-gauge, half-inch needle on a tuberculin syringe (BD, Franklin Lakes, N.J., USA) followed by introduction of a blunt 33-gauge needle on a Hamilton syringe. GenTeal gel, 0.3% (Novartis) was applied to the corneal surface and a glass coverslip was laid onto this interface for visualization through the microscope to guide the needle into the vitreous cavity without retinal or lenticular perforation. Extreme care was taken with this visualization technique to confirm that no retinal perforation occurred.

For studies evaluating activity of the hGRK1 promoter in C57BL/6 mice, 7.5×10⁹ vg of AAV2-based vectors, 8.5×10¹⁰ vg of AAVS(singleY-F) or 5.3×10⁹ vg of AAVS(doubleY-F) were delivered. For studies evaluating the CBA promoter in C57BL/6 mice, all vectors were delivered at a concentration of 1.5×10¹⁰ vg. To evaluate transduction of vectors containing microRNA target sequence in C57BL/6 mice, a concentration of 1.5×10¹⁰ vg was used. All Rho-GFP mice were injected intravitreally with 1.5×10⁹ vg.

Fundoscopy: At 4-weeks' post-injection, fundoscopy was performed using a using a Micron III camera (Phoenix Research Laboratories, Pleasanton, Calif., USA). Bright field, green fluorescent and red fluorescent images were taken to visualize retinal health, GFP expression and mCherry expression, respectively. Exposure settings were constant between experiments.

Retinal dissociation and FACS analysis: 4-weeks' post-injection, Rho-GFP retinas were harvested and dissociated with the papain dissociation system (Worthington Biochemical Corporation, Lakewood, N.J., USA). FACS analysis was used to quantify the percentage of cells that were GFP positive (PRs), mCherry positive (any retinal cells transduced with vector) and both GFP and mCherry positive (PRs transduced by vector). The percentage of mCherry positive PRs was calculated as the ratio of cells both GFP and mCherry positive relative to total GFP-positive PRs.

Immunohistochemistry (IHC): Immediately after fundoscopy, eyes were enucleated and tissue was prepared for cryoprotection and sectioning as previously described (Boye et al., 2011). Briefly, after rinsing with 1X PBS, sections were incubated with 0.5% Triton X-100 for 1 hr followed by a 30-min incubation with a blocking solution of 1% bovine serum albumin (BSA). Retinal sections were then incubated overnight at 4° C. in a rabbit polyclonal antibody raised against GFP (University of Florida, Gainesville, Fla., USA) diluted in 0.3% Triton X-100/1% BSA at 1:1,000. The following day, sections were rinsed with 1× PBS and incubated for 1 hr at room temperature in anti-rabbit IgG secondary antibody Alexa-Fluor488™ (Invitrogen, Corp., Eugene, Oreg., USA) diluted in 1× PBS at 1:500. Finally, sections were counterstained with 4′,6′-diamidino-2-phenylindole (DAPI) for 5 min at room temperature. Retinal sections were imaged using a fluorescent Axiophot® microscope (Zeiss, Thornwood, N.Y., USA) as previously described (Boye et al., 2011). Images were captured at 5×, 20× and 40×. Exposure settings were consistent across images at each magnification.

A semi-quantitative comparison of the number of GFP-positive photoreceptors was made between eyes injected intravitreally with either AAV2(quadY-F+T-V)-hGRK1-GFP or AAV2(quadY-F+T-V)-CBA-GFP (identical titers) by counting GFP-positive photoreceptors in representative sections. Low magnification (merged, 10×) and high magnification (40×) images were taken. Cell counts were made in 4 anatomically-matched areas of each representative retina. Each respective area was uniform in size by virtue of magnification (40×) and contained on average 30 columns of photoreceptor cell bodies. Results were plotted in Sigma Plot for graphical presentation.

Results

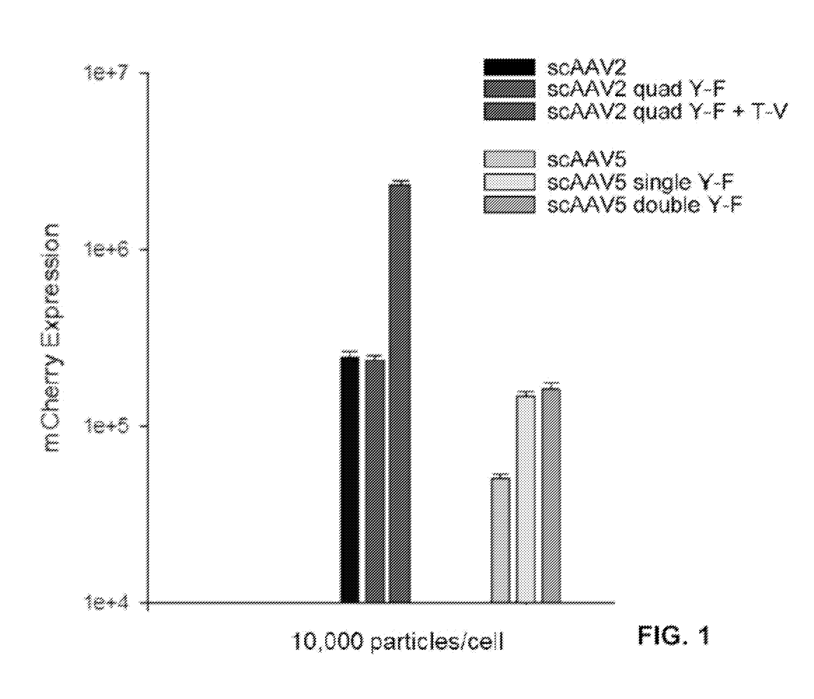

Quantification of in vitro transduction efficiency: 661W mouse cone PR cells were infected with unmodified or capsid mutated, self-complimentary AAV vectors containing the smCBA promoter driving mCherry in order to quantify relative transduction efficiencies of all vectors. FACS analysis provided a measure of relative transduction efficiency (mCherry expression) across samples. FIG. 1 shows mCherry expression, in arbitrary units, for each capsid tested; scAAV2, scAAV2(quadY-F), scAAV2(quadY-F+T-V), scAAV5, scAAV5(singleY-F), and scAAV5(doubleY-F). This screen revealed that AAV2(quadY-F+T-V) transduced cone cells most efficiently. Increases in AAV2(quadY-F+T-V) mediated mCherry expression were ˜10 fold above the scAAV2 baseline (FIG. 1).

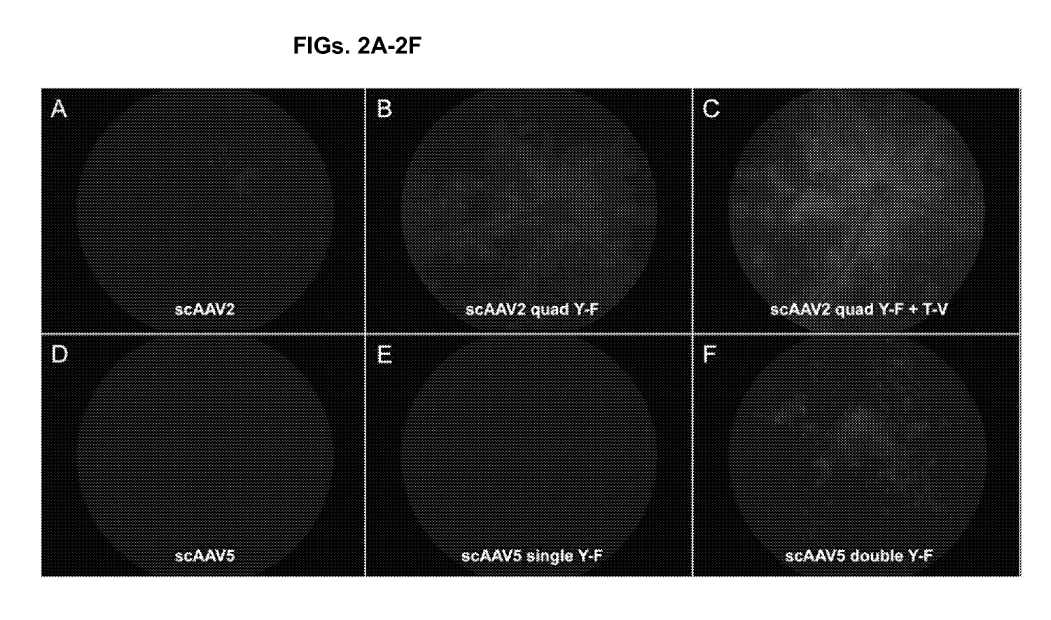

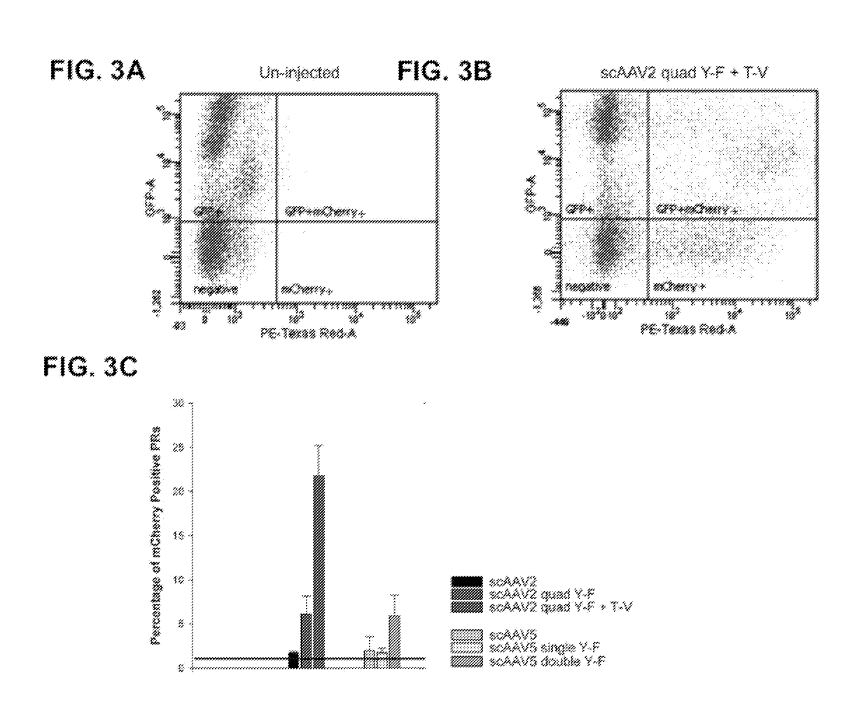

Quantification of in vivo transduction efficiency: Following in vitro screening, identical vectors were evaluated for their relative ability to transduce PRs in vivo following intravitreal injection in 1 month old, heterozygote Rho-GFP mice (1.5×10⁹ vector genomes (vg) delivered). Fundoscopy at 4-weeks' post-injection showed qualitatively that mCherry expression was enhanced with addition of capsid mutations to each serotype (FIG. 2A, FIG. 2B, FIG. 2C, FIG. 2D, FIG. 2E, and FIG. 2F). Rho-GFP mouse retinas injected intravitreally with scAAV2(quadY-F+T-V)-smCBA-mCherry exhibited the highest qualitative levels of mCherry expression (FIG. 2C). Levels of transgene expression achieved following intravitreal injection of scAAV2(quadY-F), and scAAV5(doubleY-F) were approximately equivalent. To quantify the relative ability of each vector to transduce PRs, intravitreally injected Rho-GFP retinas were dissociated and FACS analysis performed. Cells were sorted into four populations: 1) non-fluorescent: indicating un-transduced, non-PR retinal cells (“negative”), 2) green fluorescent only: indicating untransduced PRs (“GFP+”), 3) red and green fluorescent: indicating transduced PRs (“GFP+mCherry+”) and 4) red fluorescent only: indicating transduced non-PR retinal cells (“mCherry+”) (FIG. 3). As shown in FIG. 3A and FIG. 3B, an un-injected Rho-GFP retina contains two populations of cells (“GFP+” representing PRs and “negative” representing non-PRs) whereas a Rho-GFP retina injected with scAAV2(quadY-F+T-V) contains all four populations of cells. The relative percentage of mCherry-positive PRs following intravitreal injection of all vectors is shown in FIG. 3C. Addition of quadY-F and quadY-F+T-V mutations to the AAV2 capsid surface resulted in ˜3.5 fold and ˜13 fold increases in the percentage of mCherry positive PRs, respectively. Unmodified scAAV2 transduced 1.7% of PRs from the vitreous whereas scAAV2(quadY-F) and scAAV2(quadY-F+T-V) transduced 6.1% and 21.8%, respectively. scAAV2(quadY-F+T-V) transduced the highest number of PRs of all vectors tested. Retinas injected with unmodified and modified AAVS-based vectors exhibited lower efficiencies of PR transduction. Consistent with fundoscopic observations, appreciable PR transduction was seen following intravitreal injection of scAAV2(quadY-F), scAAVS(doubleY-F). The percent of mCherry positive PRs in retinas injected with scAAVS, scAAV5(singleY-F) and scAAVS(doubleY-F) was 2.0%, 1.7% and 5.9%, respectively. It was also found that quantitative comparisons could be made using this methodology at just 1-week post-intravitreal injection with scAAV2-based vectors. While fewer total PRs expressed detectable levels of mCherry at this early time point, the pattern remained the same, with scAAV2(quadY-F+T-V) mediating the highest levels of transgene expression in PRs.

Qualitative analysis of photoreceptor transduction: With the intention to restrict transgene expression to PRs following intravitreal delivery of AAV, the PR-specific hGRK1 promoter was incorporated into unmodified and capsid-mutated vectors. To evaluate vectors that are relevant for treatment of inherited retinal disease (e.g., those that can accommodate promoter and transgene sequence likely too large to package as self-complementary AAV), all vectors in this set of experiments were single stranded, e.g., non self-complementary. Representative fundus images of C57BL/6 mice and their immunostained retinal sections taken 4 weeks post-intravitreal injection with AAV2, AAV2(quadY-F) and AAV2(quadY-F+T-V) are shown in FIG. 4A, FIG. 4B, FIG. 4C, FIG. 4D, FIG. 4E, and FIG. 4F (7.5×10⁹ vg delivered for all vectors.) Consistent with the quantification results (shown in FIG. 3A-FIG. 3C), very few PRs expressed GFP following intravitreal injection of AAV2 or AAV2(quad Y-F) (FIG. 4A and FIG. 4B). However, robust GFP expression was seen in the PRs following injection of AAV2(quadY-F+T-V) (FIG. 4F). AAV2(quadY-F+T-V)-mediated transgene expression was evident in PRs throughout the retina rather than in one specific location. This representative section, in conjunction with surgical observations and fundoscopy support the premise that the injection procedure did not involve retinal perforation and was, in fact, intravitreal (FIG. 2A-FIG. 2F). Although reports have shown that the hGRK1 promoter has exclusive activity in rods and cones of mouse and non-human primate when incorporated into subretinally-delivered AAV (Boye et al., 2012; Khani et al., 2007) hGRK1-mediated transgene expression was observed in ganglion cells of injected mice (FIG. 4D and FIG. 4F).

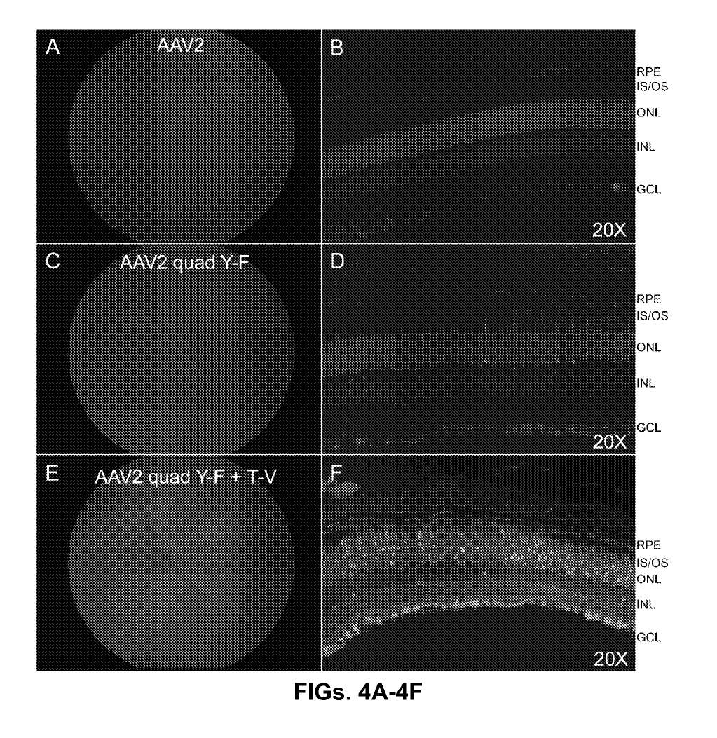

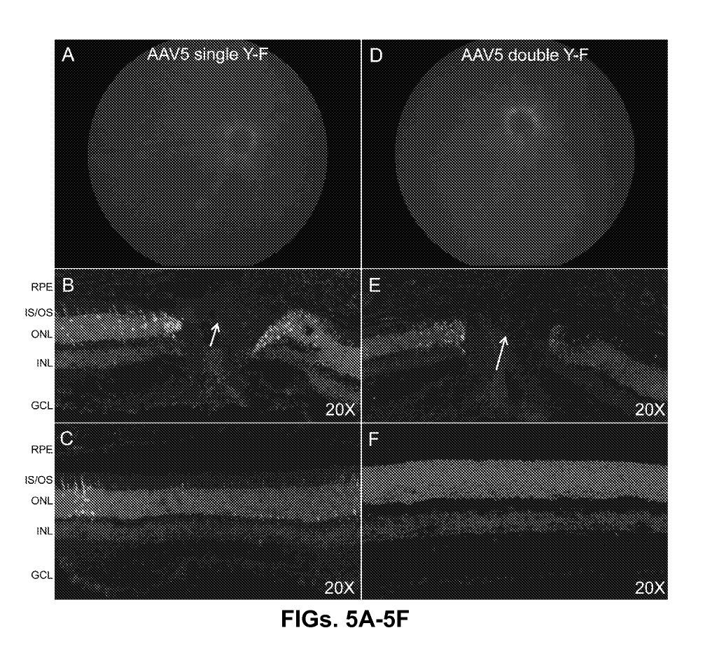

In vivo quantification data in Rho-GFP mice revealed relatively low levels of PR transduction following intravitreal delivery of 1.5×10⁹ vg of AAVS-based vectors (FIG. 3A-FIG. 3C). Therefore, in order to maximize expression and qualitatively analyze general transduction patterns, higher titers of AAVS- and AAV8-based vectors were used for the following experiments. For analysis of AAVS(singleY-F) and AAVS(doubleY-F) vectors 8.5×10¹⁰ vg and 5.3×10⁹ vg were delivered, respectively. Fundus images paired with fluorescent images of retinal cross-sections show minimal PR transduction following intravitreal injection of AAVS(singleY-F) and AAVS(doubleY-F) (FIG. 5A, FIG. 5B, FIG. 5C, FIG. 5D, FIG. 5E and FIG. 5F). A pattern of peripapillary tropism was evident, with PRs around the optic nerve exhibiting the most prominent transgene expression (FIG. 5A, FIG. 5B, FIG. 5D and FIG. 5E). PR transduction was found in scattered peripheral retinal sections of AAVS(singleY-F)-injected eyes (FIG. 5C), with expression typically found near the retinal vasculature.

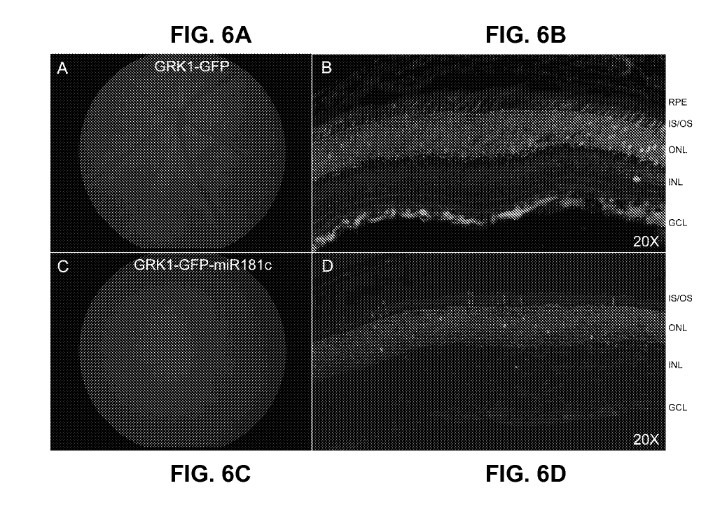

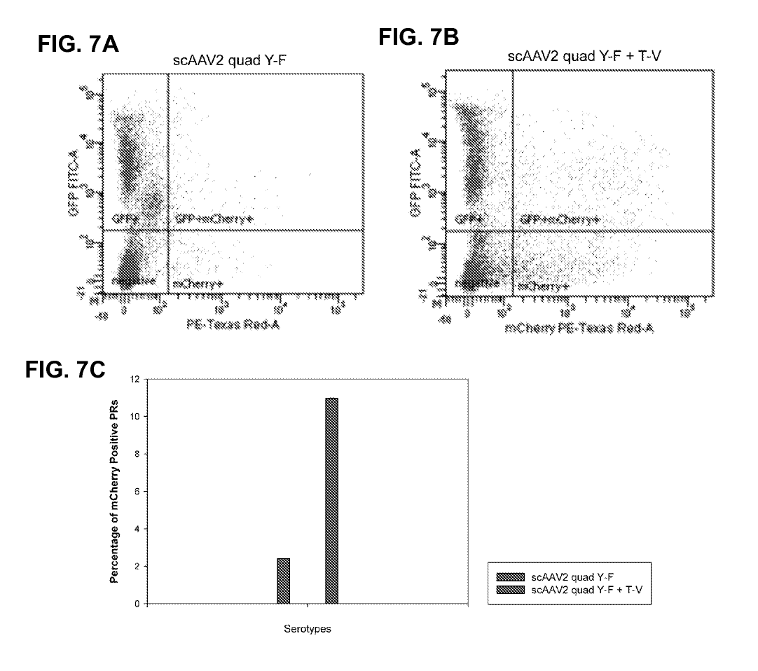

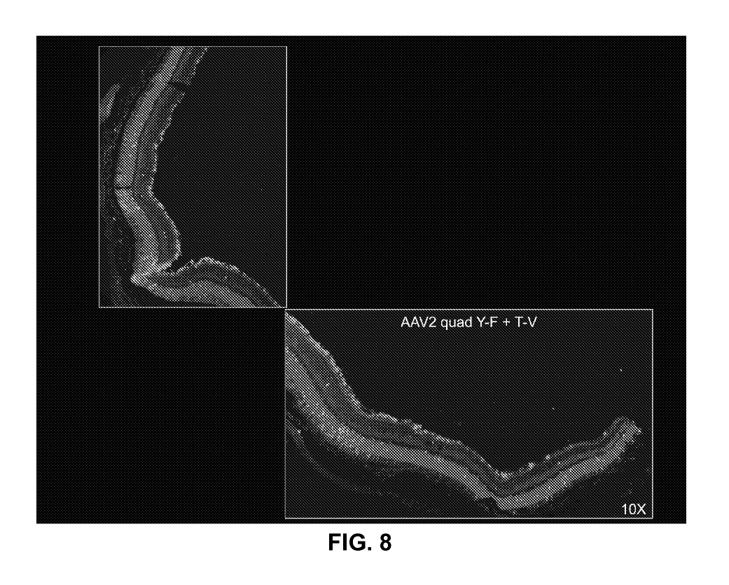

MicroRNA-mediated regulation of transgene expression: In order to mitigate the observed off-target transgene expression in ganglion cells following intravitreal delivery of hGRK1-containing AAV vectors, a target sequence was incorporated for miR181, an miRNA shown to be expressed exclusively in ganglion cells and inner retina into the disclosed AAV vectors (Atlas of miRNA distribution: http://mirneye.tigem.it/) immediately downstream of GFP, similar to previous reports (Karali et al., 2011). The intended effect was to degrade vector derived transcripts and inhibit synthesis of viral-mediated protein in all cells of the retina except PRs. Both hGRK1-GFP and hGRK1-GFP-miR181c were packaged in AAV2(quadY-F+T-V) and delivered intravitreally to C57BL/6 mice (1.5×10¹⁰ vg). At 4-weeks' post-intravitreal injection, fundoscopy and IHC on frozen retina cross-sections revealed that addition of miR181c to the vector construct did eliminate off-target expression (FIG. 6A, FIG. 6B, FIG. 6C, and FIG. 6D). Although hGRK1-GFP-miR181c-mediated GFP expression was exclusive to PRs, it was also appreciably decreased.

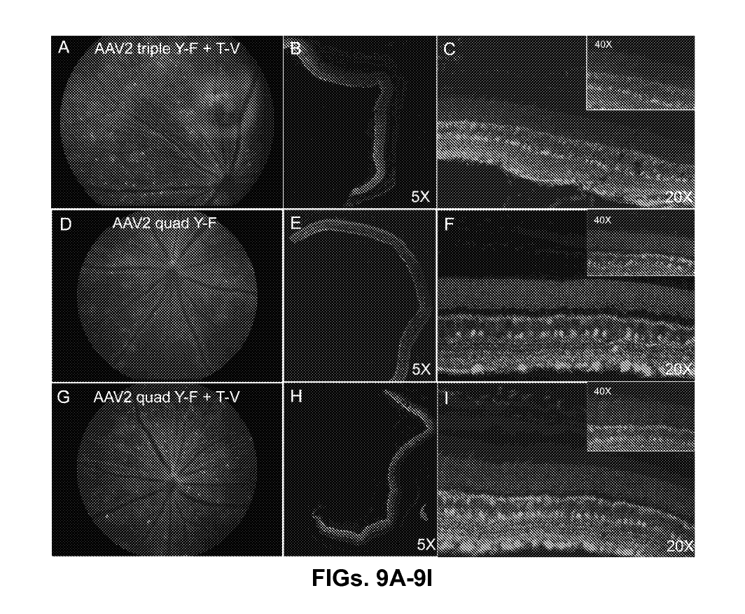

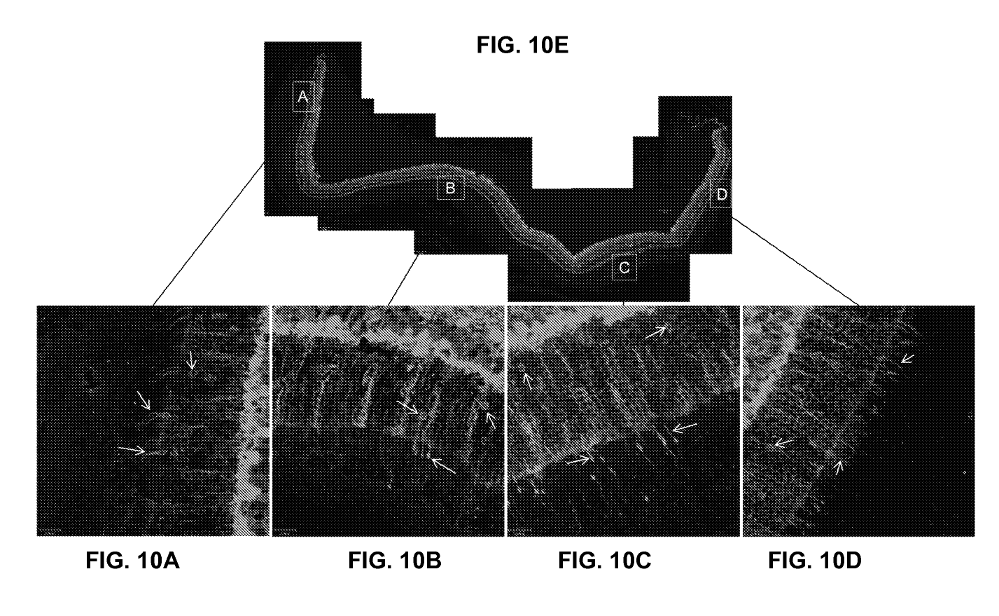

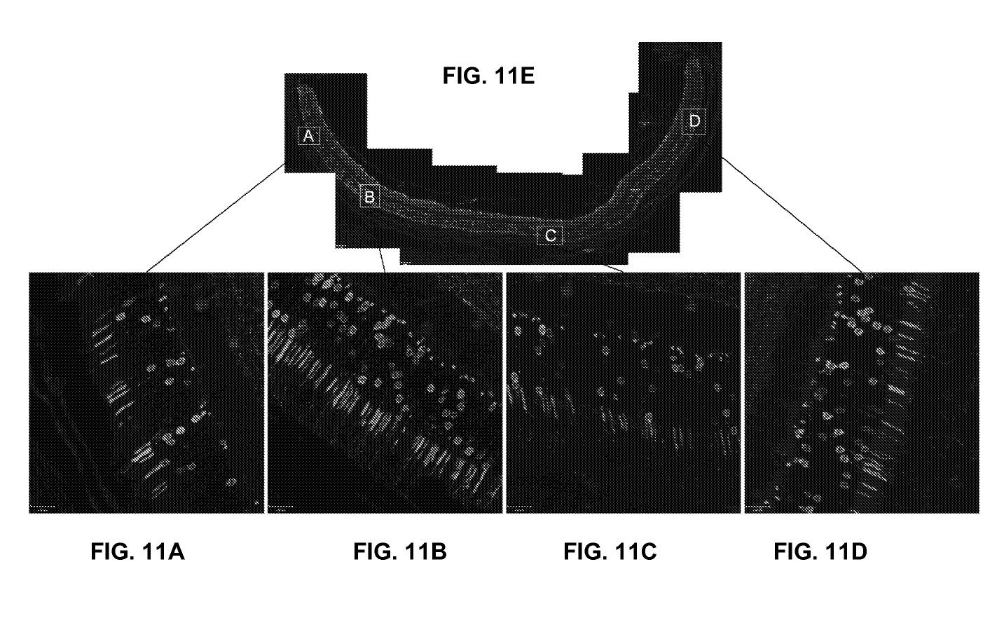

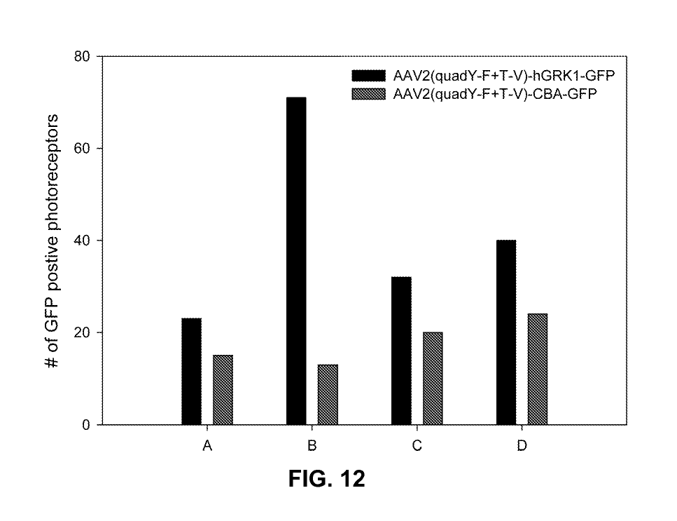

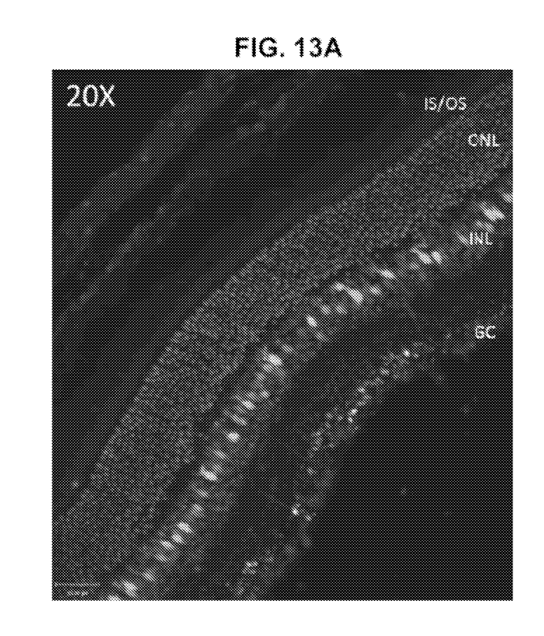

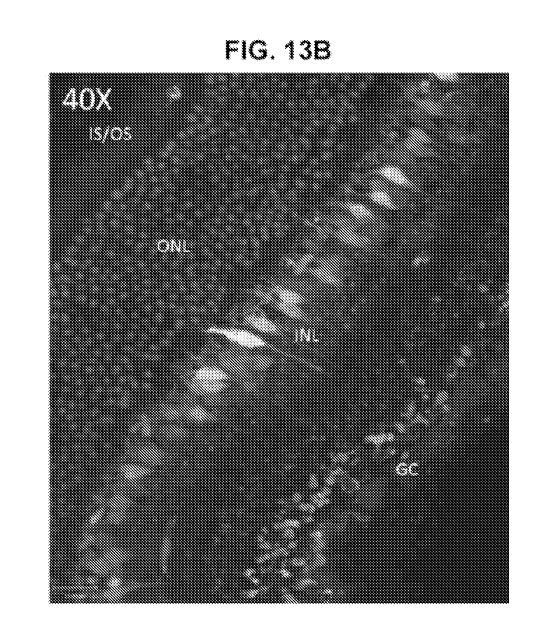

Qualitative analysis of serotype tropism: Because mutations in genes expressed in retinal cell types other than PRs can also cause or result in retinal degeneration, the ubiquitous CBA promoter was incorporated into vectors to ascertain what other retinal cells types were targeted following intravitreal injection of the strongest capsid-mutated vectors (FIG. 9A, FIG. 9B, FIG. 9C, FIG. 9D, FIG. 9E, FIG. 9F, FIG. 9G, FIG. 9H, and FIG. 9I). All vectors were delivered intravitreally at a concentration of 1.5×10¹⁰ vg. AAV2(quadY-F) and AAV2(quadY-F+T-V) vectors were chosen for testing based on their performance in the PR-targeting experiments described above. AAV2(tripleY-F+T-V) was chosen based on the documented efficiency of AAV2(tripleY-F) in multiple in vitro and in vivo settings (Petrs-Silva et al., 2011; Ryals et al., 2011). All AAV2-based vectors mediated robust, pan-retinal GFP expression (FIG. 4A, FIG. 4B, FIG. 4D, FIG. 4E, and FIG. 4F), with GFP found throughout the inner and middle retina (FIG. 4B, FIG. 4C, FIG. 4E, and FIG. 4F). AAV2(quadY-F+T-V)- and AAV2(tripleY-F+T-V)-mediated GFP expression was also seen in PR cells bodies (FIG. 9C and FIG. 9I). A semi-quantitative comparison of photoreceptor transduction following injection of either AAV2(quadY-F+T-V)-CBA-GFP or AAV2(quadY-F+T-V)-hGRK1-GFP was made by counting GFP-positive photoreceptors in 4 representative areas of retina injected with each respective vector. Whole eyecups (merged 10× images) and high magnification (40×) images of representative sections are shown in FIG. 10A, FIG. 10B, FIG. 10C, FIG. 10D, the composite shown in FIG. 10E; and in FIG. 11A, FIG. 11B, FIG. 11C, FIG. 11D, and the composite shown in FIG. 11E. GFP positive photoreceptors in retinas injected with AAV2(quadY-F+T-V)-CBA-GFP were distinguished from Muller glia by counting GFP positive cell bodies and outer segments (white arrows, FIG. 13A and FIG. 13B). A comparison of cell counts is presented in FIG. 12. GFP-positive photoreceptors were more prevalent throughout the retinas of AAV2(quadY-F+T-V)-hGRK1-GFP-injected mice.

The development of viral vectors capable of efficiently transducing PRs via a less invasive delivery method than the previously utilized subretinal injection route would be a critical advance in retinal gene therapy. In recent years, focus has been placed on identifying novel AAV capsid variants that exhibit increased transduction efficiency and/or altered tropism. To this end, two methodologies have been employed; rational mutagenesis and directed evolution. These approaches have led to identification of novel capsids with increased transduction efficiency (Petrs-Silva et al., 2009) altered tropism (Petrs-Silva et al., 2011; Klimczak et al., 2009; Pulicherla and Asokan, 2011) and the ability to evade recognition by the immune system (Li et al., 2012). “Rational mutagenesis” describes a knowledge-based approach to manipulating the viral capsid to develop customized vectors with distinctive features. Rational mutagenesis of surface-exposed tyrosine, threonine and lysine residues results in increased transduction by decreasing phosphorylation and subsequently reducing ubiquitination and proteosomal degradation of the AAV capsid (Thong et al., 2008; Aslanidi et al., 2013; Gabriel et al., 2013). It was previously shown that Y-F mutations on the AAV2, AAV8 and AAV9 capsid surface led to increased transduction and altered transduction profiles relative to unmodified vectors following both sub-retinal and intravitreal delivery (Petrs-Silva et al., 2009; Petrs-Silva et al., 2011). Later studies showed that incorporation of these mutations led to more pronounced rescue in animal models of inherited retinal disease (Boye et al., 2011; Pang et al., 2012) and in one case, conferred therapy in a particularly aggressive mouse model that was refractory to treatment using an unmodified parent serotype (Pang et al., 2011). Directed evolution can select for desired characteristics without a priori knowledge of the physical determinants, allowing identification of novel vectors that exhibit desired, specific tropisms (Bartel et al., 2012). Directed evolution has been applied to select AAV variants from combinatorial libraries that demonstrate a diverse range of cellular tropisms in vivo relative to their parent serotypes (Bartel et al., 2012). In the retina, this technology was used to identify a variant capable of specifically transducing Muller cells via the vitreous (Klimczak et al., 2009).

With the goal to develop vectors capable of transducing PRs via intravitreal delivery, extracellular determinants of viral transduction must also be considered. The internal limiting membrane (ILM) which defines the border between the retina and vitreous acts as a physical and biological barrier to AAV transduction following intravitreal injection in rodent and non-human primate retina (Dalkara et al., 2009; Yin et al., 2011). It has been shown that AAV2 and AAV8 attach to the ILM and accumulate at the vitreoretinal junction, with AAV2 exhibiting the most robust attachment (Dalkara et al., 2009). However, only AAV2 mediated detectable transgene expression in the inner retina (Dalkara et al., 2009). AAV2 binds heparan sulfate proteoglycan (HSPG) which is abundant in the ILM (Summerford and Samulski, 1998; Chai and Morris, 1994), while AAV8 binding involves the laminin receptor, which may mediate a weaker interaction with this structure (Akache et al., 2006). This example illustrates that addition of Y-F and T-V mutations to the AAV8 capsid modestly improves its ability to transduce inner/middle/outer retina following intravitreal injection demonstrating the importance of both extracellular and intracellular barriers to transduction. Standard AAV5 fails to attach or accumulate at the ILM (Dalkara et al., 2009), likely because it relies on sialic acid for initial binding, a monosaccharide absent from the ILM (Kaludov et al., 2001; Cho et al., 2002). Removal of this physical barrier with protease, however, led to robust gene expression in various cells of the retina, including PRs and RPE (Dalkara et al., 2009). Similar to AAV8, here it is shown that addition of Y-F mutations to the AAV5 capsid surface only modestly improves its ability to transduce outer retina following intravitreal delivery. Taken together, it is clear that the cellular receptors of the parent AAV serotype play a key role in influencing vector interaction with this vitreoretinal interface. These results are consistent with findings that AAV2-based vectors have the highest affinity for the ILM (Dalkara et al., 2009) suggesting that, as of now, capsid mutants based on this serotype have the highest potential for targeting transgene to PRs via the vitreous. As the capsid biology of AAV8, a strong transducer of PRs in situ, becomes known, an approach that capitalizes on respective receptor biology of AAV2 and AAV8 may yield improved variants (Raupp et al., 2012).

An ideal approach would be to identify variants with the ability to reach/target the tissue of interest through manipulation of capsid receptor biology. This variant would then be further modified to account for intracellular trafficking. A method that utilizes directed evolution to find variants with increased affinity for PRs that can subsequently be enhanced by incorporation of the appropriate combination of Y-F and or T-V mutations may ultimately be the most successful strategy, particularly if powerful quantitative assays can be used to rapidly and accurately assess in vivo vector properties. Methods for quantifying vector transduction efficiency were previously shown in a biologically relevant, PR cell line (Ryals et al., 2011). This example provides reliable in vivo assay for quantifying transduction efficiencies of intravitreally-delivered AAV vectors in mouse PRs. The quantitative results here correlated well to qualitative fundoscopic observations. Quantitative findings could be obtained as early as one-week post-injection and that, although fewer total cells appear transduced at this early time point relative to 4 weeks post-injection, the pattern and relative efficiencies of vectors remained the same.

Of all the vectors that have been tested to date, the most robust in vivo expression of PRs was noted following intravitreal delivery of AAV2(quadY-F+T-V)-smCBA-GFP. Approximately 22% of PRs expressed detectable levels of transgene following intravitreal injection with this capsid mutant. To what extent transduction of 22% of PRs is capable of preserving retinal structure and/or restoring visual function to an animal model of IRD is yet to be determined. Likewise, whether further improvements in transduction efficiency of the AAV2(quadY-F+T-V) can be achievable by additional mutagenesis requires further investigation. Evidence suggests that directed mutagenesis of additional threonine, lysine and serine residues, all of which are more abundant on the AAV2 capsid surface than tyrosine, and similarly reduce phosphorylation/proteosomal degradation of capsid, may further augment AAV-mediated transgene expression (Gabriel et al., 2013). It is expected that this approach will have a finite maximum. However, it is important to note that the transduction efficiency of capsid mutant vectors varies with the target tissue as well as the profile and activity levels of kinases involved in AAV capsid phosphorylation (Aslanidi et al., 2012). Additionally, it has yet to be determined whether initially non surface-exposed residues that become available for phosphorylation in later steps of cellular processing (during conformational changes of the capsid) may also be mutated to improve transduction efficiency.

When considering intravitreal delivery of AAV vector intended to transduce distal PRs, emphasis must be placed on avoiding off-target transgene expression. Consistent with previous reports (Boye et al., 2012; Khani et al., 2007), it was found that the hGRK1 promoter drove strong transgene expression in PRs. Unexpectedly, off-target expression was also noted in retinal ganglion cells. Previous studies evaluating GRK1 promoter activity in retina have utilized AAV serotypes with poor tropism for retinal ganglion cells, namely AAV5 and AAV8 (Boye et al., 2012; Boye et al., 2011; Khani et al., 2007; Boye et al., 2010; Beltran et al., 2010). Therefore, it is unlikely (even in the event such vectors were delivered to the vitreous) that transduction of retinal ganglion cells would have occurred. When a parent serotype was used with strong affinity for retinal ganglion cells (AAV2), and delivered high titer vector to the vitreous, GRK1 promoter activity in retinal ganglion cells was apparent. Because GRK1 has been shown to promote strong gene expression in both rods and cones of primate retina with no expression in middle retina or retinal pigment epithelium (Boye et al., 2012) the inventors sought to address specifically the observed expression in retinal ganglion cells. An attempt to reduce this off-target expression was used by incorporating four tandem sequences complimentary to an inner/middle retina-specific miRNA into AAV vectors. A microRNA expression atlas of the mouse eye (Karali et al., 2010) indicates that miR-181c is highly expressed in retinal ganglion cells and middle retina and absent in photoreceptors in P60 mouse (http://mirneye.tigem.it/view_state.php?state=P60&mirna=mmu-miR-181c). Incorporation of miR-181c repeat sequence resulted in ablation of expression in retinal ganglion cells; however, it also appreciably reduced expression of transgene in PRs.

This example provides evidence for the continued development of AAV-based vectors to treat various forms of PR-mediated inherited retinal disease using a surgically-less-invasive intravitreal injection technique.

Once it was established that AAV2(quadY-F+T-V) was the most effective vector for transducing all retinal cell types (including bipolar cells) following intravitreal injection in mice, it was reasoned that specific cell types could targeted by incorporating cell specific promoters. Incorporation of the novel “Ple155” promoter (derived from the regulatory region of purkinje cell protein 2 (PCP2), a protein expressed in ON bipolar cells) into the AAV2(quadY-F+T-V) multi capsid mutant vector led to transgene expression exclusively in ON bipolar cells of mice intravitreally injected with this vector, as evidenced by colocalization of GFP and PCP2 expression in immunostained retinal cross sections from intravitreally injected wild type mice (FIG. 13). Robust and highly selective GFP expression was seen throughout the retinas of injected eyes.

The sequence of the Ple115 promoter is provided below:

Ple 155 promoter sequence (1651 bps):

(SEQ ID NO: 12)
cagcagattgaagaagccctcctggtctggggagcccgcctggggacaga
ctcgctcagtctggttggcccttcagcttgggggcccctccccaaacttc
ctagccagcttgctcacaccctgaccccggggcctgccgtccccacttcc
tcagctctcaccatggtccccgccgatcctctctgcagagctgttctgaa
tgagacatgagtctccttcccaatcccggcccccgccccaggggccctgg
ccagcagtgccacttcacgtggtaccgcttcaagggacaggctccgatgc
gtgtcccgccgtcctagattggggcctgatgagtgtggcctgggagctgg
gacacgaatcagggaaacatggcccaggagctacccccaggtcccagcat
ctcccatcaataggggtccacacggagagccctgccctctgccctggggc
ctggcactcagacccccaagcccaccagcccctttctacagccacaactg
ggtcagggggtcctaggagactcactcgttaattaggtgccctacaaact
aattagtcttgtcaatcatgggctctgagaccttgagctgggggtggggt
gggggcagggccctctcacctcggcacaggggcctgagccttcctccgtc
ttctcctcctgatccggacacttcattggcatagagggagagagtgtgaa
cttggccctttgtggaacagaggaggctcgggcagaggtggtgatagtgc
agcccattcattctgagatgaaacttccactggtttccgtaaagacgtct
tggggagggaagggaaggggatggggacctcccagtggtatcccctgctt
gggcactgagggaaagccacagtggctcggggtaaaaggcagggacatcc
tctccccgcctgcctctgtccccagggagtctcgcctcctgttcccacct
ggggctagggtgatagaggagaggagatagctcaacctggcatttaggtg
gtgtgggaacaggagaccccagactttcttgttttggggtctggggcagg
caaccaggctccagggacagtgagttgaaggaagggtggctgggagaccc
cttgacttgctgccaaggagacagagctggagctagggtggcgggtggtg
tctgaggcaggtgcagagagggagggagggaaggggcctttgactccaac
ctcctttttctttaccgactgcaggtggcagctgcccttccaggagccag
tgggggaacctgggtggctgggtggggacacctgcaagtcctccctaagc
cagctaccaccctacactgttggcctcccttctccaactgtggggacgct
gctcaggccttttgtgacatcacacctgagagtccctggggtccagtcat
tgctgctgggcacagcgaggtccaagctcaggtcgccctgccccctaccc
accatgccagatccagcatcgttgtgggcaaacaattatctggatgatct
ttatggggcttaagcttgggtgggagcagatggggcatgagctggggatt
tggggatggggggaatccacacccccacgtcctggacgtttaaaaggccc
tctctggcactgggccggggcagaggccagcagaaaagtgactggagtcc
a

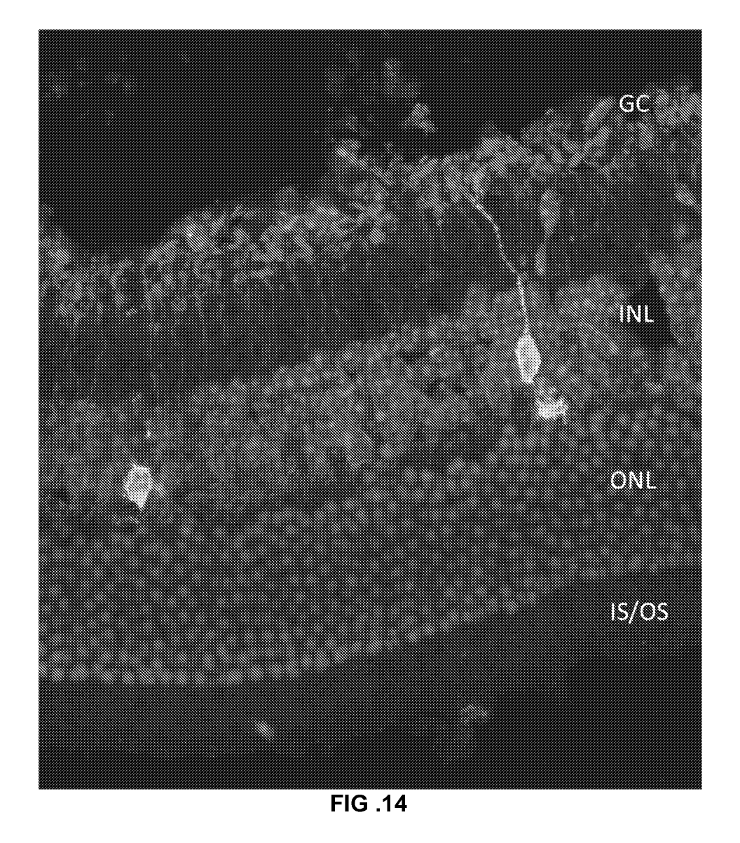

Having developed an AAV serotype/cellular promoter combination to drive robust and highly selective transgene expression in ON bipolars, the inventors next asked whether it was possible to express nyctalopin (nyx) in this cell type following intravitreal injection. Mutations in nyctalopin (nyx) are associated with X-linked congenital stationary night blindness (XCSNB). The specific nyctalopin transgene chosen to deliver contained a YFP tag (for detection via IHC as there are currently no reliable antibodies directed against nyx). Its expression via transgenesis has been confirmed previously (Gregg et al., 2007). A representative image of retina from a wild type mouse intravitreally injected with AAV2(quadY-F+T-V)-Ple155-YFP/nyx and stained with antibodies against GFP and PKCα (an ON bipolar specific marker) revealed ON bipolar-specific expression of nyx (YFP) (FIG. 14).

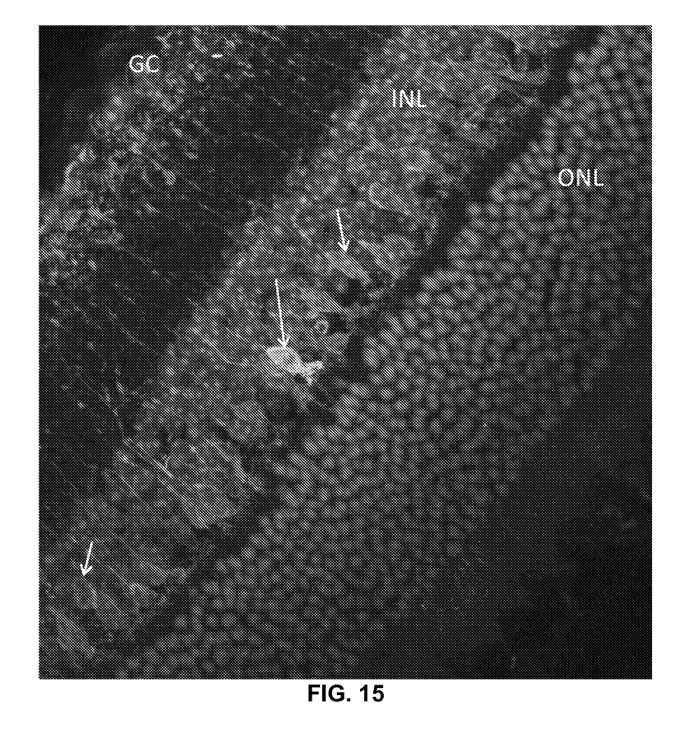

With the ultimate goal to use this serotype/promoter combination to deliver therapeutic transgene to animal models of congenital stationary night blindness, AAV2(quadY-F+T-V)-Ple155-YFP/nyx was injected into nyx/nob (Nyx^{nob}) mice. The nyx/nob (Nyx^{nob}) mouse, a well-charaterized model of XCSNB (Gregg et al., 2003; Pardue et al., 1998), carries a frameshift mutation in nyx, exhibits lack of b-wave on electroretinogram (indicative of a signaling problem in bipolar cells) and lacks retinal degeneration. Representative retinal cross sections from intravitreally injected nyx/nob (Nyx^{nob}) mice exhibit ON-bipolar specific expression of nyx (YFP) as evidenced by immunostaining for GFP and PKCcL (FIG. 15). While AAV2(quadY-F+T-V)-Ple155-mediated nyx expression was less robust than GFP, it was still restricted to the target cell (ON bipolars). Using the same vector, it was shown that nyx expression was also restricted to ON bipolars following delivery of identical vector to the subretinal space.

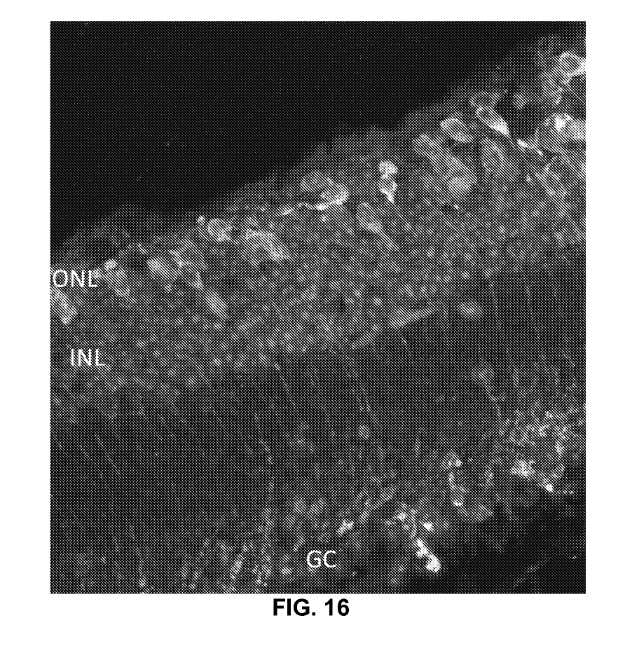

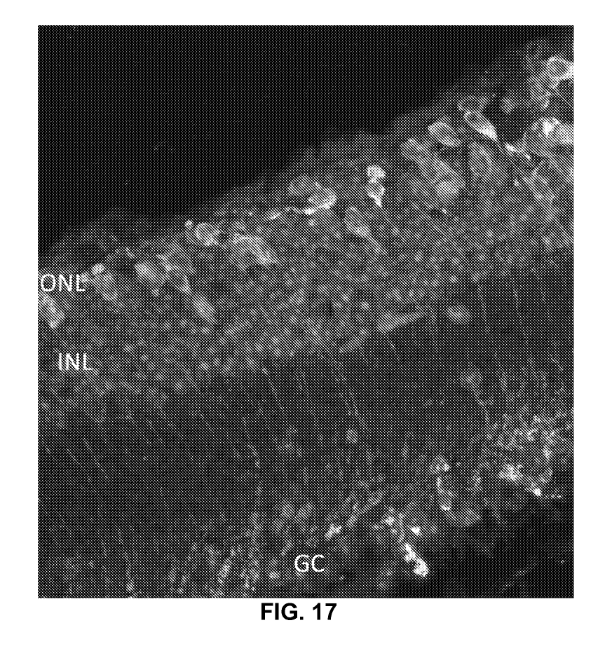

In order to determine whether the degenerative state of the retina would alter the tropism of AAV2(quadY-F+T-V)-Ple155-GFP (in other words, would one find off-target expression in a retina that is actively degenerating?), this vector was injected either subretinally or intravitreally into rd16 mice. This well-characterized mouse model of Leber congenital amaurosis (LCA) harbors a mutation in Cep290 and exhibits rapid retinal degeneration (only one row of photoreceptor nuclei remain at 5 weeks of age) (Chang et al., 2006). These results also showed that AAV2(quadY-F+T-V)-Ple155-mediated GFP expression was found in ON bipolar cells of rd16 mice after injection either via the subretinal (FIG. 16) or intravitreal space (FIG. 17). These results demonstrated that this serotype/promoter combination is effective for delivering transgene to ON bipolars in mouse retinas that are either preserved over time, or are actively degenerating.

### EXAMPLE 2

### Further Data Related to ON Bipolar Cell Transgene Delivery

Introduction: Congenital Stationary Night Blindness (CSNB) is an inherited retinal disorder characterized by the inability to see in low light conditions. Patients also have difficulties seeing in daylight conditions due to a high frequency of myopia, nystagmus, and strabismus. In the complete form of this disease (CSNB1), signaling from photoreceptors to ON bipolar cells (ON BCs) is disrupted due to mutations in genes encoding post synaptic proteins involved in the metabotropic glutamate receptor 6 (mGluR6) G-protein coupled cascade. Despite this signaling dysfunction, the retinas of CSNB1 patients and mouse models do not degenerate. One such gene, NYX, encodes Nyctalopin and its mutated form is associated with X-linked CSNB1. Without functional NYX, TRPM1 cation channel is mislocalized and cannot be gated. Using AAV2(quadY-F+T-V)-Ple155-YFP/NYX, the below study established an intravitreal method of delivery for AAV-mediated transduction of ON BCs. The potential of this tool to restore mGluR6-mediated signaling in ON BCs in a model of XCSNB 1, the Nyx^{nob} mouse, was evaluated.

Methods: A construct containing an ON BC specific promoter, Ple155, driving YFP_mNyx was packaged into an AAV2(quadY-F+T-V). Postnatal day 2 or 30 (P2 or P30) Nyx^{nob} mice were intravitreally injected with 6.5×10¹² VG. Electoretinograms (ERG) and whole cell patch clamp responses were recorded from injected and control eyes ˜4 weeks post injection. Retinas were examined using immunohistochemistry and confocal microscopy.

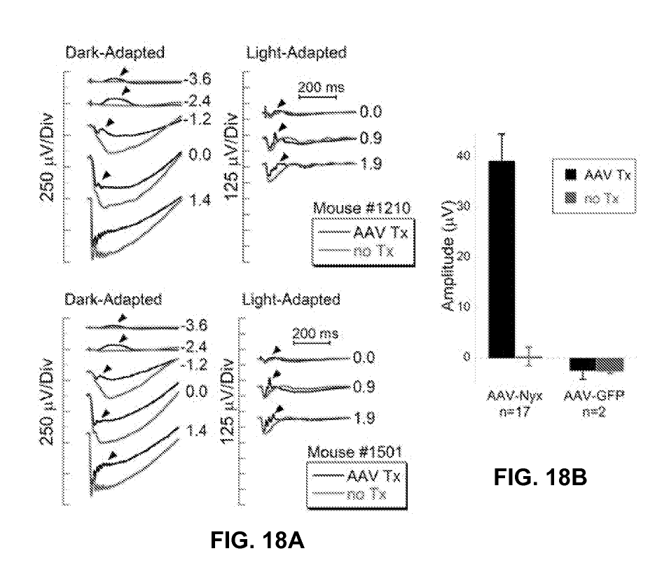

Results: Electroretinograms (ERGs) were recorded in Nyx^{nob} mice treated with AAV in one eye to assess bipolar cell functionality. FIG. 18A shows a complete set of electroretinograms (ERGs) recorded from two Nyx^{nob} mice, each of which were treated with AAV2(quadY-F+T-V)-Ple155-YFP/nyx in one eye while the other eye was untouched. The dark-adapted ERGs obtained from untreated eyes were comparable to other no b-wave (nob) models (Pardue & Peachey, 2014), with an overall negative polarity comprised of slow PIII which, in response to high luminance stimuli, is preceded by a faster a-wave (FIG. 18A). In comparison, dark-adapted ERGs obtained from AAV-treated eyes had a clear positive component that was observed in all AAV-treated eyes but never in an untreated eye (FIG. 18A). This component was most readily seen in response to the lower luminance stimuli, where the Nyx^{nob} ERG is essentially flat or comprised of only a small amplitude slow PIII. FIG. 18B summarizes the magnitude of this positive component obtained to −2.4 log cd s/m² stimuli, which averaged almost 40 μV across the 17 mice examined. When the same analysis was applied to the responses of untreated eyes, the average was not different than zero, and a very similar result was obtained when two mice treated with AAV-GFP were studied.

The dark-adapted responses indicate that AAV treatment restored function to rod DBCs (depolarizing bipolar cells). To determine if cone DBCs were similarly affected, ERGs to stimuli superimposed upon a steady rod-desensitizing adapting field were also recorded. Under these conditions, Nyx^{nob} ERGs were somewhat more complex, as are responses of other mutants for DBC genes (Pardue & Peachey 2014). This reflects in part the additional contribution of cone HBCs (hyperpolarizing bipolar cells) to the cone ERG signal (Shirato et al., 2008). The two sets of cone ERG waveforms shown in FIG. 18A indicate that AAV treatment resulted in the presence of a reproducible positive signal that was not present in untreated eyes or in eyes treated with AAV-GFP.

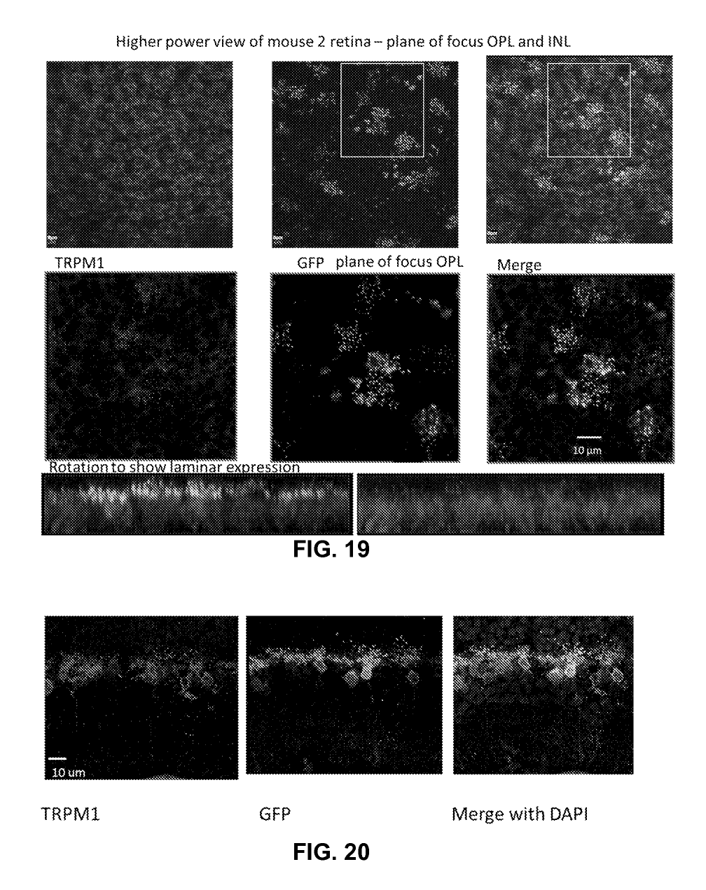

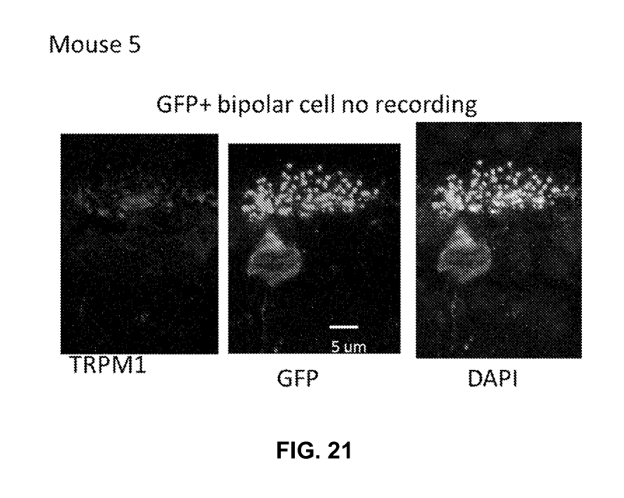

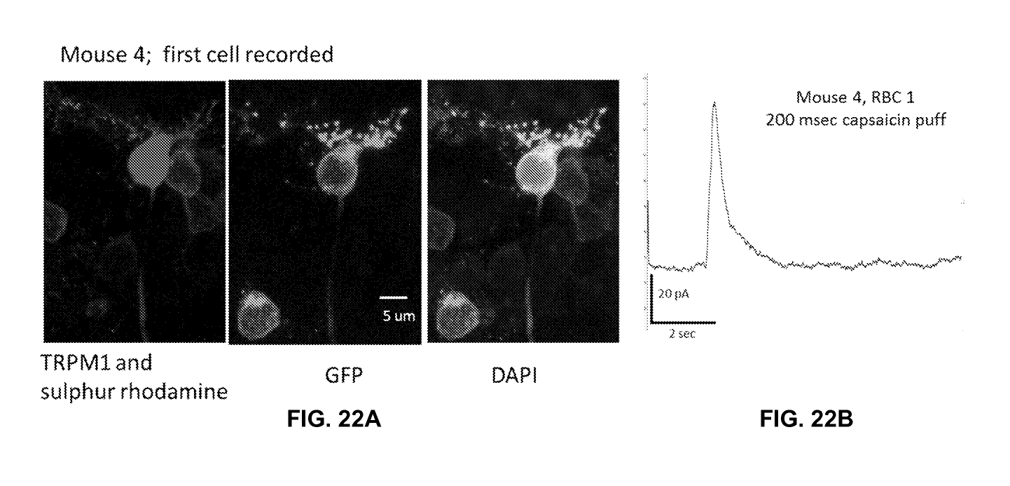

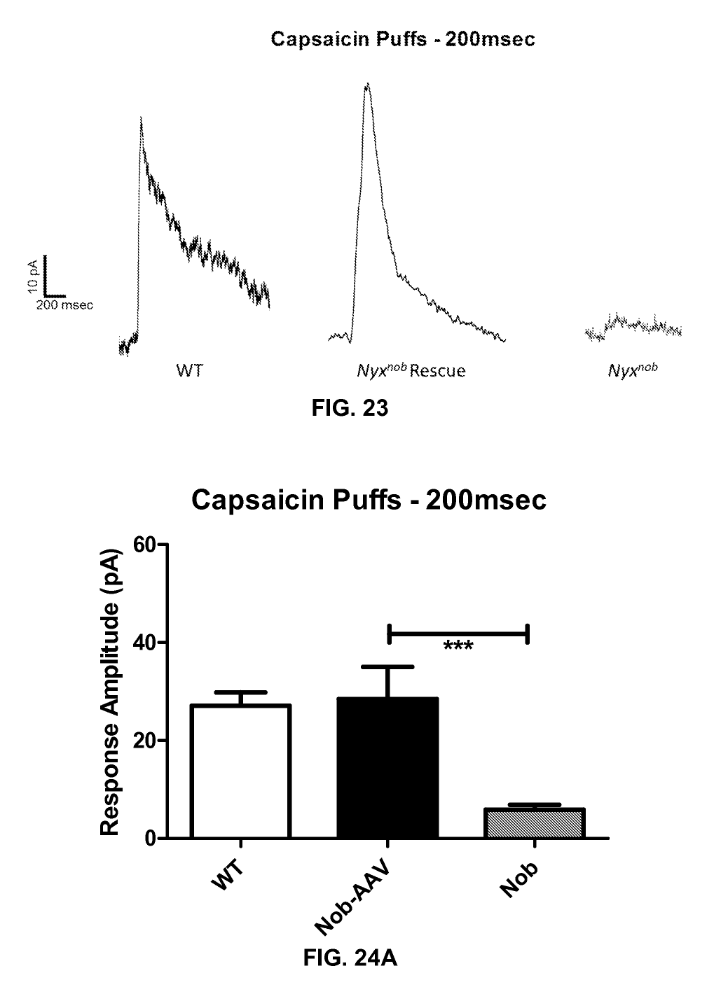

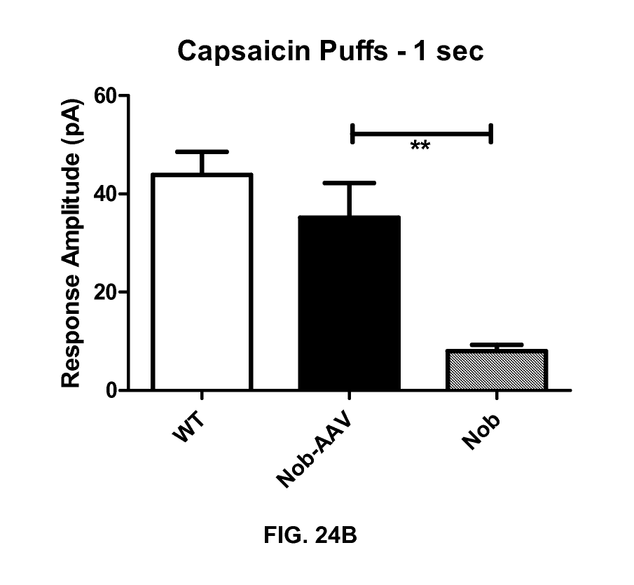

Further studies were undertaken to assess the functionality of ON bipolar cells (BCs) in retinas from Nyx^{nob} mice injected at postnatal day 2 (P2) with AAV2(quadY-F+T-V)-Ple155-YFP/NYX. These mice had restored retinal function as assessed by ERG. These retinas were stained for TRPM1 (the ion channel important for glumate signaling in ON BCs). In untreated bipolar cells, TRPM1 failed to localize to the dendritic tips (remained in biosynthetic membranes instead, FIG. 19). Colocalization of GFP antibody (GFP antibody was used to label the YFP tag attached to the NYX protein) and TRPM1 was observed in the AAV-treated ON bipolar cells (FIGS. 19 and 20). This indicated that gene therapy resulted in proper localization of the channel. Single cell recordings were then used to evaluate whether the cells transduced with AAV2(quadY-F+T-V)-Ple155-YFP/NYX had restored functionality. Using microscopy, one of the cells that was recorded from was identified (as it was filled with Sulphur Rhodamine and labeled with GFP, FIGS. 21 and 22A). A sample trace of the capsaicin (glutamate receptor agonist) response in that cell is shown in FIG. 22B. The traces showed that glutamate signaling was restored in AAV-treated cells but not the untreated cells and that their behavior was indistinguishable from wild-type cells, whereas glutamate signaling in untreated cells remained defective (FIG. 23 and FIGS. 24A and B).

These results show that eyes injected at P2 with AAV showed robust NYX expression exclusively in ON BCs, restoration of scotopic and photopic ERG b-waves and gating of the TRPM1 channel both directly and via the mGluR6 cascade (capsaicin and CPPG responses).

### EXAMPLE 3

### Injection of AAV2(quadY-F+T-V) into Non-Human Primate Retinas

Non-human primates were injected with two different AAV vectors either alone or in combination:

a. AAV2(quadY-F+T-V)-hGRK1-mCherry

b. AAV2(quadY-F+T-V)-CBA-GFP

The methodology for each animal is described in more detail below:

Animal 1: Right eye—AAV2(QuadY-F+T-V)-hGRK1-mCherry and CBA-GFP combined, Subretinal, 100 ul peripheral bleb (1×10e11 vg per vector) and Intravitreal, 200 ul (2×10e11 vg per vector); Left eye—AAV2(QuadY-F+T-V)-CBA-GFP, Intravitreal, 100 ul (3×10e11 vg).

Animal 2: Right eye—AAV2(QuadY-F+T-V)-hGRK1-mCherry and CBA-GFP combined, Subretinal, 100 ul peripheral bleb (1×10e11 vg per vector) and Intravitreal, 200 ul (2×10e11 vg per vector); Left eye—AAV2(quadY-F+T-V)-hGRK-mCherry, Intravitreal, 100 ul (3×10e12 vg).

The purpose of the study was study transduction in both the peripheral and central retina. To achieve peripheral transduction, the vector(s) were delivered to the subretinal space. As the injection device was withdrawn, vector was also placed in the vitreous. The animals were then imaged 4 weeks post-injection, 8 weeks post injection, and 10.5 weeks post injection.

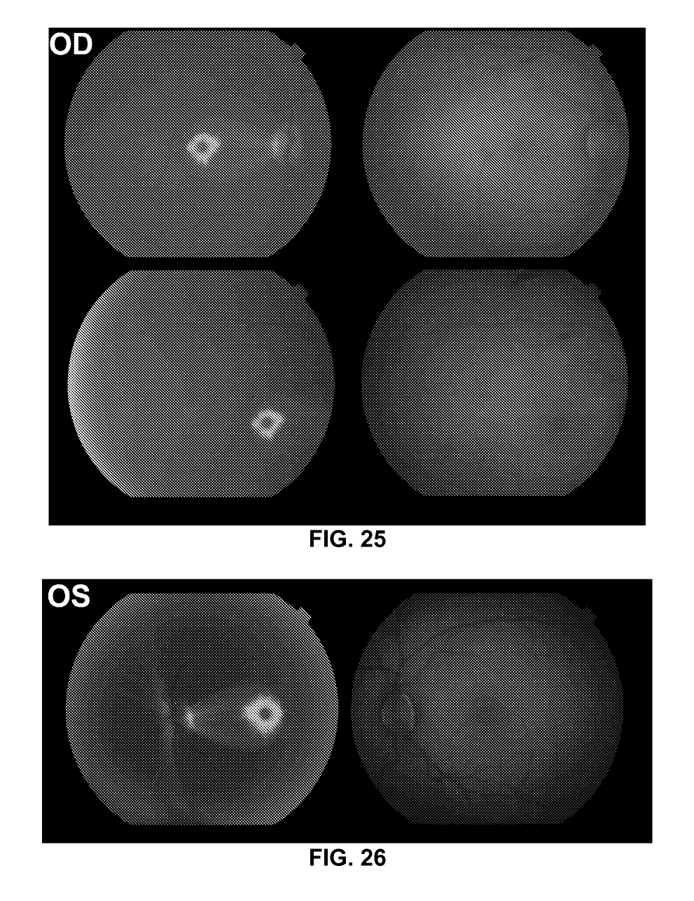

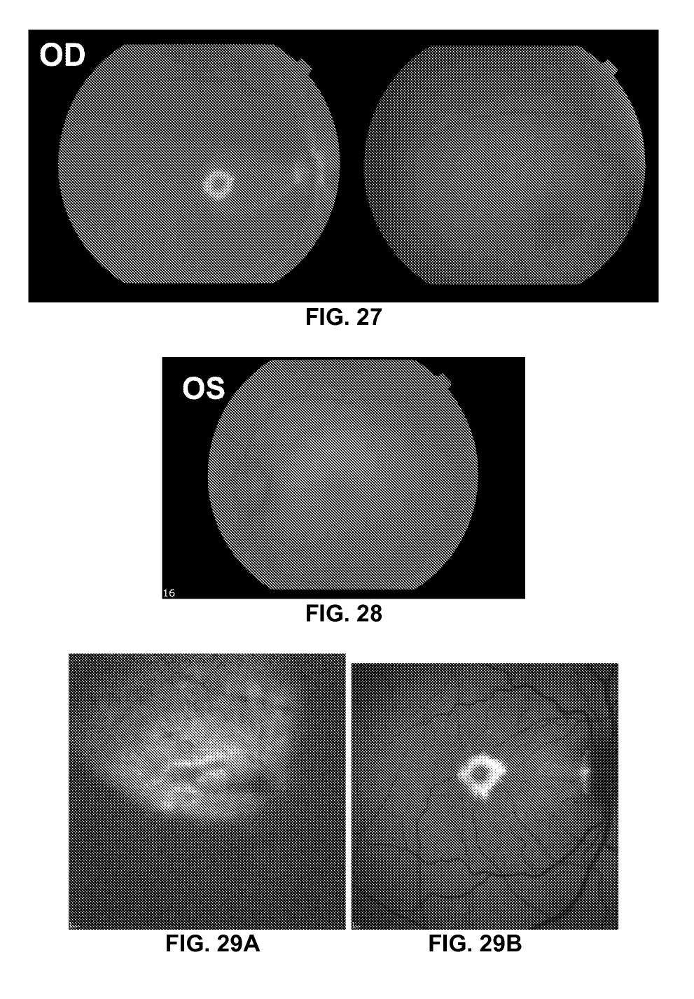

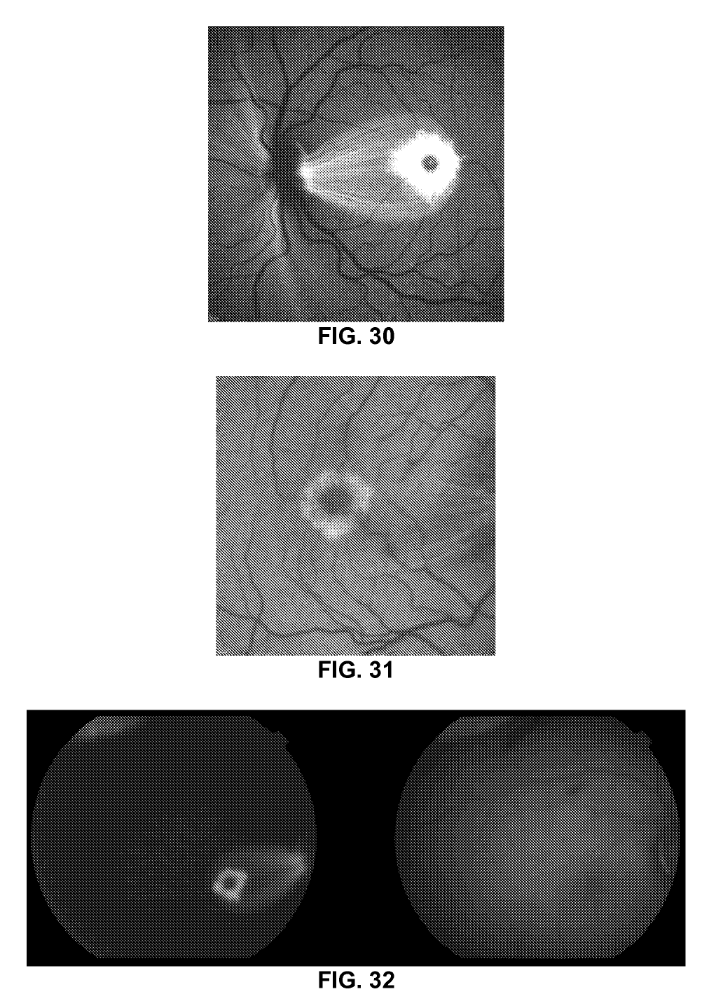

Strong GFP expression was observed in the retinal ganglion cell ring around the foveal pit and in the foveal cone photoreceptors at 4 weeks post injection (FIGS. 25-27). GFP expression was observed both in the eyes that received the subretinal+intravitreal injection and in the eyes that received the intravitreal injection alone. Expression of GFP was still visible at 8 weeks post injection (FIGS. 29-31).

At 4 weeks, mCherry expression was not yet visible in the eyes (FIGS. 25-28), which was attributed to the strength and speed of the hGRK1 promoter relative to CBA. At 10.5 weeks post injection, mCherry expression was visible via fundoscopy in the peripheral subretinal bleb and the fovial pit (FIG. 32).

These results show that AAV2(QuadY-F+T-V) can also infect the retina of non-human primates.

### REFERENCES

The following references, to the extent that they provide exemplary procedural or other details supplementary to those set forth herein, are specifically incorporated herein by reference:

Acland, G M et al., “Gene therapy restores vision in a canine model of childhood blindness,” Nat. Genet., 28:92-95 (2001).

Akache, B et al., “The 37/67-kilodalton laminin receptor is a receptor for adeno-associated virus serotypes 8, 2, 3, and 9,” J. Virol., 80:9831-9836 (2006).

Ali, R R et al., “Gene transfer into the mouse retina mediated by an adeno-associated viral vector,” Hum. Mol. Genet., 5:591-594 (1996).

Allocca, M et al., “Novel adeno-associated virus serotypes efficiently transduce murine photoreceptors,” J. Virol., 81:11372-11380 (2007).

Al-Ubaidi, M R et al., “Proteomics profiling of the cone photoreceptor cell line, 661W,” Adv. Exp. Med. Biol., 613:301-311 (2008).

Aslanidi, G V et al., “Optimization of the capsid of recombinant 1 adeno-associated virus 2 (AAV2) vectors: The final threshold?” PLoS One, 8(3):e59142 (2013).

Aslanidi, G V et al., “High-efficiency transduction of human monocyte-derived dendritic cells by capsid-modified recombinant AAV2 vectors,” Vaccine, 30:3908-3917 (2012).

Auricchio, A et al., “Exchange of surface proteins impacts on viral vector cellular specificity and transduction characteristics: the retina as a model,” Hum. Mol. Genet., 10:3075-3081 (2001).

Bainbridge, J W et al., “Effect of gene therapy on visual function in Leber's congenital amaurosis,” N. Engl. J. Med., 358:2231-2239 (2005).

Bartel, M A et al., “Directed evolution of novel adeno-associated viruses for therapeutic gene delivery,” Gene Ther., 19:694-700 (2012).

Beltran, W A et al., “rAAV2/5 gene-targeting to rods:dose-dependent efficiency and complications associated with different promoters,” Gene Ther., 17:1162-1174 (2010).

Bennett, J et al., “Stable transgene expression in rod photoreceptors after recombinant adeno-associated virus-mediated gene transfer to monkey retina,” Proc. Natl. Acad. Sci. USA, 96:9920-9925 (1999).

Boye, S E et al., “The human thodopsin kinase promoter in an AAVS vector confers rod- and cone-specific expression in the primate retina,” Hum. Gene Ther., 23:1101-1115 (2012).

Boye, S E et al., “Functional and behavioral restoration of vision by gene therapy in the guanylate cyclase-1 (GC1) knockout mouse,” PLoS One, 5:e11306 (2010).

Boye, S L et al., “Long-term preservation of cone photoreceptors and restoration of cone function by gene therapy in the guanylate cyclase-1 knockout (GC1KO) mouse,” Invest. Ophthalmol. Vis. Sci., 52:7098-7108 (2011).

Burger, C et al., “Recombinant AAV viral vectors pseudotyped with viral capsids from serotypes 1, 2, and 5 display differential efficiency and cell tropism after delivery to different regions of the central nervous system,” Mol. Ther., 10:302-317 (2004).

Chai, L and Morris, J E, “Distribution of heparan sulfate proteoglycans in embryonic chicken neural retina and isolated inner limiting membrane,” Curr. Eye Res., 13:669-677 (1994).

Chang B et al., “In-frame deletion in a novel centrosomal/ciliary protein CEP290/NPHP6 perturbs its interaction with RPGR and results in early-onset retinal degeneration in the rd16 mouse,” Hum. Mol. Genet., 15(11):1847-57 (2006).

Cho, E Y et al., “Expression pattern of glycoconjugates in rat retina as analysed by lectin histochemistry,” Histochem. J., 34:589-600 (2002).

Cideciyan, A V et al., “Human RPE65 gene therapy for Leber congenital amaurosis: persistence of early visual improvements and safety at 1 year,” Hum. Gene Ther., 20:999-1004 (2009).

Dalkara, D et al., “Inner limiting membrane barriers to AAV-mediated retinal transduction from the vitreous,” Mol. Ther., 17:2096-2102 (2009).

Daya, S and Berns, K I, “Gene therapy using adeno-associated virus vectors,” Clin. Microbiol. Rev., 21:583-593 (2008).

Gabriel, N et al., “Bio-engineering of AAV-2 capsid at specific serine, threonine or lysine residues improves its transduction efficiency in vitro and in vivo,” Hum. Gene Ther., Methods, 24(2):80-93 (2013).

Gregg R G et al., “Identification of the gene and the mutation responsible for the mouse nob phenotype,” Invest. Ophthalmol. Vis. Sci., 44:378-384 (2003).

Gregg R G et al., “Nyctalopin expression in retinal bipolar cells restores visual function in a mouse model of complete X-linked congenital stationary night blindness,” J. Neurophysiol., 98(5):3023-33 (2007).

Haire, S E et al., “Light-driven cone arrestin translocation in cones of postnatal guanylate cyclase-1 knockout mouse retina treated with AAV-GC1,” Invest. Ophthalmol. Vis. Sci., 47:3745-3753 (2006).

Jacobson, S G et al., “Safety of recombinant adeno-associated virus type 2-RPE65 vector delivered by ocular subretinal injection,” Mol. Ther., 13:1074-1084 (2006).

Jacobson, S G et al., “Gene therapy for leber congenital amaurosis caused by RPE65 mutations: safety and efficacy in 15 children and adults followed up to 3 years,” Arch. Ophthalmol., 130:9-24 (2012).

Kaludov, N et al., “Adeno-associated virus serotype 4 (AAV4) and AAVS both require sialic acid binding for hemagglutination and efficient transduction but differ in sialic acid linkage specificity,” J. Virol., 75:6884-6893 (2001).

Karali, M et al., “MicroRNA-restricted transgene expression in the retina,” PLoS One, 6:e22166 (2011).

Karali, M et al., “miRNeye: a microRNA expression atlas of the mouse eye,” BMC Genomics, 11:715 (2010).

Khani, S C et al., “AAV-mediated expression targeting of rod and cone photoreceptors with a human rhodopsin kinase promoter,” Invest. Ophthalmol. Vis. Sci., 48:3954-3961 (2007).

Klimczak, R R, “A novel adeno-associated viral variant for efficient and selective intravitreal transduction of rat Muller cells,” PLoS One, 4:e7467 (2009).

Li, C et al., “Single amino acid modification of adeno-associated virus capsid changes transduction and humoral immune profiles,” J. Virol., 86:7752-7759 (2012).

Lotery, A J et al., “Adeno-associated virus type 5: transduction efficiency and cell-type specificity in the primate retina,” Hum. Gene Ther., 14:1663-1671 (2003).

Maguire, A M et al., “Safety and efficacy of gene transfer for Leber's congenital amaurosis,” N. Engl. J. Med., 358:2240-2248 (2008).

Matsumoto, B et al., “Topographic variations in the rabbit and primate internal limiting membrane,” Invest. Ophthalmol. Vis. Sci., 25:71-82 (1984).

Mussolino, C et al., “AAV-mediated photoreceptor transduction of the pig cone-enriched retina,” Gene Ther., 18:637-645 (2011).

Pang, J J et al., “Long-term retinal function and structure rescue using capsid mutant AAV8 vector in the rd10 mouse, a model of recessive retinitis pigmentosa,” Mol. Ther., 19:234-242 (2011).

Pang, J J et al., “AAV-mediated cone rescue in a naturally occurring mouse model of CNGA3-achromatopsia,” PLoS One, 7:e35250 (2012).

Pardue M T et al., “A naturally occurring mouse model of X-linked congenital stationary night blindness,” Invest. Ophthalmol. Vis. Sci., 39:2443-2449 (1998).

Petersen-Jones, S M et al., “AAV retinal transduction in a large animal model species: comparison of a self-complementary AAV2/5 with a single-stranded AAV2/5 vector,” Mol. Vis., 15:1835-1842 (2009).

Petrs-Silva, H et al., “Novel properties of tyrosine-mutant AAV2 vectors in the mouse retina,” Mol. Ther., 19:293-301 (2011).

Petrs-Silva, H et al., “High-efficiency transduction of the mouse retina by tyrosine-mutant AAV serotype vectors,” Mol. Ther., 17:463-471 (2009).

Pulicherla, N and Asokan, A, “Peptide affinity reagents for AAV capsid recognition and purification,” Gene Ther., 18:1020-1024 (2011).

Rabinowitz, J E et al., “Cross-packaging of a single adeno-associated virus (AAV) type 2 vector genome into multiple AAV serotypes enables transduction with broad specificity,” J. Virol., 76:791-801 (2002).

Raupp, C et al., “The threefold protrusions of adeno-associated virus type 8 are involved in cell surface targeting as well as postattachment processing,” J. Virol., 86:9396-9408 (2012).

Ryals, R C et al., “Quantifying transduction efficiencies of unmodified and tyrosine capsid mutant AAV vectors in vitro using two ocular cell lines,” Mol. Vis., 17:1090-1102 (2011).

Stieger, K et al., “Subretinal delivery of recombinant AAV serotype 8 vector in dogs results in gene transfer to neurons in the brain,” Mol. Ther., 16:916-923 (2008).

Summerford, C and Samulski, R J, “Membrane-associated heparan sulfate proteoglycan is a receptor for adeno-associated virus type 2 virions,” J. Virol., 72:1438-1445 (1988).

Tan, E et al., “Expression of cone-photoreceptor-specific antigens in a cell line derived from retinal tumors in transgenic mice,” Invest. Ophthalmol. Vis. Sci., 45:764-768 (2004).

Vandenberghe, L H et al., “Cone and rod transduction with alternative AAV serotypes in the macula of non-human primates,” Conference proceedings, Association for Research in Vision and Ophthalmology (ARVO), Ft. Lauderdale, Fla. (2011).

Vandenberghe, L H et al., “Dosage thresholds for AAV2 and AAV8 photoreceptor gene therapy in monkey,” Sci. Transl. Med., 3:88ra54 (2011).

Weber, M et al., “Recombinant adeno-associated virus serotype 4 mediates unique and exclusive long-term transduction of retinal pigmented epithelium in rat, dog, and nonhuman primate after subretinal delivery,” Mol. Ther., 7:774-781 (2003).

Wensel, T G et al., “Rhodopsin-EGFP knock-ins for imaging quantal gene alterations,” Vision Res., 45:3445-3453 (2005).

Wright, A F et al., “Photoreceptor degeneration: genetic and mechanistic dissection of a complex trait,” Nat. Rev. Genet., 11:273-284 (2010).

Yang, G S et al., “Virus-mediated transduction of murine retina with adeno-associated virus: effects of viral capsid and genome size,” J. Virol., 76:7651-7660 (2002).

Yin, L et al., “Intravitreal injection of AAV2 transduces macaque inner retina,” Invest. Ophthalmol. Vis. Sci., 52:2775-2783 (2011).

Thong, L et al., “Next generation of adeno-associated virus 2 vectors: point mutations in tyrosines lead to high-efficiency transduction at lower doses,” Proc. Natl. Acad. Sci. USA, 105:7827-7832 (2008).

Zolotukhin, S et al., “Production and purification of serotype 1, 2, and 5 recombinant adeno-associated viral vectors,” Methods, 28:158-167 (2002).

It should be understood that the examples and embodiments described herein are for illustrative purposes only and that various modifications or changes in light thereof will be suggested to persons skilled in the art and are to be included within the spirit and purview of this application and the scope of the appended claims. All references, including publications, patent applications and patents, cited herein are hereby incorporated by reference to the same extent as if each reference was individually and specifically indicated to be incorporated by reference and was set forth in its entirety herein. Recitation of ranges of values herein are merely intended to serve as a shorthand method of referring individually to each separate value falling within the range, unless otherwise indicated herein, and each separate value is incorporated into the specification as if it were individually recited herein.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents that are chemically and/or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. An adeno-associated viral (AAV) particle comprising:
(a) a recombinant adeno-associated viral (rAAV) vector polynucleotide that comprises a nucleic acid segment that encodes a diagnostic or therapeutic agent operably linked to an ON bipolar cell-specific promoter that is capable of expressing the nucleic acid segment in one or more middle retinal neuron cells of a mammalian eye; and
(b) a modified capsid protein, wherein the modified capsid protein comprises at least a first non-native amino acid at a position that corresponds to a surface-exposed amino acid residue in the wild-type AAV2 capsid protein, and further wherein the transduction efficiency of a virion comprising the modified capsid protein is higher than that of a virion comprising a corresponding, unmodified wild-type capsid protein.

2. The AAV particle of claim 1, wherein the modified capsid protein comprises three or more non-native amino acid substitutions at positions corresponding to three distinct surface-exposed amino acid residues of the wild-type AAV2 capsid protein as set forth in SEQ ID NO:2; or to three distinct surface-exposed amino acid residues corresponding thereto in any one of the wild-type AAV1, AAV3, AAV4, AAV5, AAV6, AAV7, AAV9, or AAV10 capsid proteins, as set forth, respectively, in SEQ ID NO:1, SEQ ID NO:3, SEQ ID NO:4, SEQ ID NO:5, SEQ ID NO:6, SEQ ID NO:7, SEQ ID NO:9, or SEQ ID NO:10, or any combination thereof.

3. The AAV particle of claim 2, wherein the non-native amino acid substitutions occur at amino acid residues:
(a) Y272, Y444, Y500, and Y730;
(b) Y272, Y444, Y500, Y700, and Y730;
(c) Y272, Y444, Y500, Y704, and Y730;
(d) Y252, Y272, Y444, Y500, Y704, and Y730;
(e) Y272, Y444, Y500, Y700, Y704, and Y730;
(f) Y252, Y272, Y444, Y500, Y700, Y704, and Y730;
(g) Y444, Y500, Y730, and T491;
(h) Y444, Y500, Y730, and S458;
(i) Y444, Y500, Y730, S662, and T491;
(j) Y444, Y500, Y730, T550, and T491;
(k) Y444, Y500, Y730, T659, and T491; or
(l) Y272, Y444, Y500, Y730, and T491,
of the wild-type AAV2 capsid protein as set forth in SEQ ID NO:2, or at equivalent amino acid positions corresponding thereto in any one of the wild-type AAV1, AAV3, AAV4, AAV5, AAV6, AAV7, AAV9, or AAV10 capsid proteins, as set forth, respectively, in SEQ ID NO:1, SEQ ID NO:3, SEQ ID NO:4, SEQ ID NO:5, SEQ ID NO:6, SEQ ID NO:7, SEQ ID NO:9, or SEQ ID NO:10, or any combination thereof.

4. The AAV particle of claim 3, comprising the amino acid substitutions Y272F, Y444F, Y500F, Y730F, and T491V in a wild-type AAV2 capsid protein, or equivalent amino acid substitutions at the corresponding residues in any one of the wild-type AAV1, AAV3, AAV4, AAV5, AAV6, AAV7, AAV9, or AAV10 capsid proteins.

5. The AAV particle of claim 1, wherein the transduction efficiency of a virion comprising the modified capsid protein is about 2- to about 50-fold higher in the one or more middle retinal neuron cells than that of a virion that comprises a corresponding, unmodified, wild-type capsid protein.

6. The AAV particle of claim 1, wherein the nucleic acid segment further comprises an enhancer, a post-transcriptional regulatory sequence, a polyadenylation signal, or any combination thereof, operably linked to the nucleic acid segment encoding the diagnostic or therapeutic agent.

7. The AAV particle of claim 1, wherein the ON Bipolar cell-specific promoter is obtained from a mammalian purkinje cell protein 2 (PCP2) regulatory region.

8. The AAV particle of claim 1, wherein the therapeutic agent is a polypeptide, a peptide, a ribozyme, a peptide nucleic acid, an siRNA, an RNAi, an antisense oligonucleotide, an antisense polynucleotide, an antibody, an antigen binding fragment, or any combination thereof.

9. The AAV particle of any one of claims 1 to 8, wherein the therapeutic agent a Nyx polypeptide.

10. A method for providing a mammal in need thereof with a therapeutically-effective amount of a selected therapeutic agent, the method comprising intravitreally administering to one or both eyes of the mammal, an amount of the AAV particle of any one of claims 1 to 9; and for a time effective to provide the mammal with a therapeutically-effective amount of the selected therapeutic agent.

11. A method for treating or ameliorating one or more symptoms of a disease, a disorder, a dysfunction, an injury, an abnormal condition, or trauma in a mammal, the method comprising, intravitreally administering to one or both eyes of the mammal in need thereof, the AAV particle of any one of claims 1 to 9, in an amount and for a time sufficient to treat or ameliorate the one or more symptoms of the disease, the disorder, the dysfunction, the injury, the abnormal condition, or the trauma in the mammal.

12. A method for expressing a nucleic acid segment in one or more retinal cells of a mammal, the method comprising: intravitreally administering to one or both eyes of the mammal the AAV particle of any one of claims 1 to 9, for a time effective to produce the therapeutic agent in the one or more retinal cells of the mammal.

13. The method of claim 12, wherein the mammal has, is suspected of having, is at risk for developing, or has been diagnosed with at least a first retinal disorder, a first retinal disease, or a first retinal dystrophy, or any combination thereof.

14. The method of claim 13, wherein the retinal disease or disorder is retinitis pigmentosa, melanoma-associated retinopathy, congenital stationary night blindness, cone-rod dystrophy, Leber congenital amaurosis, or late stage age-related macular degeneration.

15. The method of claim 12, wherein the mammal is a neonate, a newborn, an infant, or a juvenile.

16. The method of claim 12, wherein production of the therapeutic agent a) preserves one or more ON bipolar cells, b) restores one or more rod- and/or cone-mediated functions, c) restores visual behavior in one or both eyes, or d) any combination thereof.

17. The method of claim 12, wherein production of the therapeutic agent persists in the one or more retinal cells substantially for a period of at least three months following a single intravitreal administration of the AAV particle into the one or both eyes of the mammal.

18. The method of claim 17, wherein production of the therapeutic agent persists in the one or more retinal cells substantially for a period of at least six months following a single intravitreal administration.

19. The method of claim 12, wherein the rAAV vector polynucleotide comprised within the AAV particle is a self-complementary rAAV (scAAV).

20. The method of claim 12, wherein the mammal is human.

21. The method of claim 12, wherein the therapeutic agent is an agonist, an antagonist, an anti-apoptosis factor, an inhibitor, a receptor, a cytokine, a cytotoxin, an erythropoietic agent, a glycoprotein, a growth factor, a growth factor receptor, a hormone, a hormone receptor, an interferon, an interleukin, an interleukin receptor, a nerve growth factor, a neuroactive peptide, a neuroactive peptide receptor, a protease, a protease inhibitor, a protein decarboxylase, a protein kinase, a protein kinsase inhibitor, an enzyme, a receptor binding protein, a transport protein or an inhibitor thereof, a serotonin receptor, or an uptake inhibitor thereof, a serpin, a serpin receptor, a tumor suppressor, a chemotherapeutic, or any combination thereof.

22. A recombinant adeno-associated viral (rAAV) vector polynucleotide that comprises a nucleic acid segment that encodes a therapeutic agent operably linked to an ON bipolar cell-specific promoter that is capable of expressing the nucleic acid segment in one or more middle retinal neuron cells of a mammalian eye.

23. The rAAV vector polynucleotide of claim 22, wherein the ON Bipolar cell-specific promoter is obtained from a mammalian purkinje cell protein 2 (PCP2) regulatory region.

24. The rAAV vector polynucleotide of claim 22 or 23, wherein the therapeutic agent a Nyx polypeptide.

25. An adeno-associated viral (AAV) particle comprising:
a modified capsid protein, wherein the modified capsid protein comprises non-native amino acid substitutions occur at amino acid residues Y272, Y444, Y500, Y730, and T491 in a wild-type AAV2 capsid protein, or equivalent amino acid substitutions at the corresponding residues in any one of the wild-type AAV1, AAV3, AAV4, AAVS, AAV6, AAV7, AAV9, or AAV10 capsid proteins.

26. The AAV particle of claim 25, comprising the amino acid substitutions Y272F, Y444F, Y500F, Y730F, and T491V in a wild-type AAV2 capsid protein, or equivalent amino acid substitutions at the corresponding residues in any one of the wild-type AAV1, AAV3, AAV4, AAV5, AAV6, AAV7, AAV9, or AAV10 capsid proteins.

27. The AAV particle of claim 25, wherein the transduction efficiency of a virion comprising the modified capsid protein is about 2- to about 50-fold higher in the one or more middle retinal neuron cells than that of a virion that comprises a corresponding, unmodified, wild-type capsid protein.

28. A nucleic acid that encodes a modified capsid protein, wherein the modified capsid protein comprises non-native amino acid substitutions occur at amino acid residues Y272, Y444, Y500, Y730, and T491 in a wild-type AAV2 capsid protein, or equivalent amino acid substitutions at the corresponding residues in any one of the wild-type AAV1, AAV3, AAV4, AAV5, AAV6, AAV7, AAV9, or AAV10 capsid proteins.

29. The nucleic acid of claim 28, wherein the modified capsid protein comprises the amino acid substitutions Y272F, Y444F, Y500F, Y730F, and T491V in a wild-type AAV2 capsid protein, or equivalent amino acid substitutions at the corresponding residues in any one of the wild-type AAV1, AAV3, AAV4, AAV5, AAV6, AAV7, AAV9, or AAV10 capsid proteins.

30. The nucleic acid of claim 28, wherein the transduction efficiency of a virion comprising the modified capsid protein is about 2- to about 50-fold higher in the one or more middle retinal neuron cells than that of a virion that comprises a corresponding, unmodified, wild-type capsid protein.


SEQUENCE LISTING (14 sequences)

SEQ ID NO: 1 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, adeno-associated virus 2 [sequence omitted]

SEQ ID NO: 3 - protein, 736 aa, Adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, Adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, Adeno-associated virus 8
  misc_feature (566)..(566): Xaa can be any naturally occurring amino acid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEXEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, Adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQANTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 11 - DNA, 18 bp, Artificial sequence
  Primer sequence
actcaccgacaggttgaa

SEQ ID NO: 12 - DNA, 1651 bp, Artificial sequence
  Ple155 promoter sequence
cagcagattgaagaagccctcctggtctggggagcccgcctggggacagactcgctcagtctggttggcccttcagcttgggggcccctccccaaacttcctagccagcttgctcacaccctgaccccggggcctgccgtccccacttcctcagctctcaccatggtccccgccgatcctctctgcagagctgttctgaatgagacatgagtctccttcccaatcccggcccccgccccaggggccctggccagcagtgccacttcacgtggtaccgcttcaagggacaggctccgatgcgtgtcccgccgtcctagattggggcctgatgagtgtggcctgggagctgggacacgaatcagggaaacatggcccaggagctacccccaggtcccagcatctcccatcaataggggtccacacggagagccctgccctctgccctggggcctggcactcagacccccaagcccaccagcccctttctacagccacaactgggtcagggggtcctaggagactcactcgttaattaggtgccctacaaactaattagtcttgtcaatcatgggctctgagaccttgagctgggggtggggtgggggcagggccctctcacctcggcacaggggcctgagccttcctccgtcttctcctcctgatccggacacttcattggcatagagggagagagtgtgaacttggccctttgtggaacagaggaggctcgggcagaggtggtgatagtgcagcccattcattctgagatgaaacttccactggtttccgtaaagacgtcttggggagggaagggaaggggatggggacctcccagtggtatcccctgcttgggcactgagggaaagccacagtggctcggggtaaaaggcagggacatcctctccccgcctgcctctgtccccagggagtctcgcctcctgttcccacctggggctagggtgatagaggagaggagatagctcaacctggcatttaggtggtgtgggaacaggagaccccagactttcttgttttggggtctggggcaggcaaccaggctccagggacagtgagttgaaggaagggtggctgggagaccccttgacttgctgccaaggagacagagctggagctagggtggcgggtggtgtctgaggcaggtgcagagagggagggagggaaggggcctttgactccaacctcctttttctttaccgactgcaggtggcagctgcccttccaggagccagtgggggaacctgggtggctgggtggggacacctgcaagtcctccctaagccagctaccaccctacactgttggcctcccttctccaactgtggggacgctgctcaggccttttgtgacatcacacctgagagtccctggggtccagtcattgctgctgggcacagcgaggtccaagctcaggtcgccctgccccctacccaccatgccagatccagcatcgttgtgggcaaacaattatctggatgatctttatggggcttaagcttgggtgggagcagatggggcatgagctggggatttggggatggggggaatccacacccccacgtcctggacgtttaaaaggccctctctggcactgggccggggcagaggccagcagaaaagtgactggagtcca

SEQ ID NO: 13 - DNA, 1446 bp, Homo sapiens
atgaaaggccgagggatgttggtcctgcttctgcatgcggtggtcctcggcctgcccagcgcctgggccgtgggggcctgcgcccgcgcttgtcccgccgcctgcgcctgcagcaccgtggagcgcggctgctcggtgcgctgcgaccgcgcgggcctcctgcgggtgccggccgagctcccgtgcgaggcggtctccatcgacctggaccggaacggcctgcgcttcctgggcgagcgagccttcggcacgctgccgtccttgcgccgcctgtcgctgcgccacaacaacctgtccttcatcacgcccggcgccttcaagggcctgccgcgcctggctgagctgcgcctggcgcacaacggcgacctgcgctacctgcacgcgcgcaccttcgcggcgctcagccgcctgcgccgcctagacctagcagcctgccgcctcttcagcgtgcccgagcgcctcctggccgaactgccggccctgcgcgaactcgccgccttcgacaacctgttccgccgcgtgccgggcgcgctgcgcggcctggccaacctgacgcacgcgcacctggagcgcggccgcatcgaggcggtggcctccagctcgctgcagggcctgcgccgcctgcgctcgctcagcctgcaggccaaccgcgtccgtgccgtgcacgctggcgccttcggggactgtggcgtcctggagcatctgctgctcaacgacaacctgctggccgagctcccggccgacgccttccgcggcctgcggcgcctgcgcacgctcaacctgggtggcaacgcgctggaccgcgtggcgcgcgcctggttcgctgacctggccgagctcgagctgctctacctggaccgcaacagcatcgccttcgtggaggagggcgccttccagaacctctcgggtctcctcgcgctgcacctcaacggcaaccgcctcaccgtgctcgcctgggtcgccttccagcccggcttcttcctgggccgcctcttcctcttccgcaacccgtggtgctgcgactgccgtctggagtggctgagggactggatggagggctccggacgtgtcaccgacgtgccgtgcgcctccccgggctccgtggccggcctggacctcagccaggtgaccttcgggcgctcctccgatggcctctgtgtggaccccgaggagctgaacctcaccacgtccagtccaggcccgtccccagaaccagcggccaccaccgtgagcaggttcagcagcctcctctccaagctgctggccccgagggtcccggtggaggaggcggccaacaccactggggggctggccaacgcctccctgtccgacagcctctcctcccgtggggtgggaggcgcgggccggcagccctggtttctcctcgcctcttgtctcctgcccagcgtggcccagcacgtggtgtttggcctgcagatggactga

SEQ ID NO: 14 - DNA, 1431 bp, Mus musculus
atgctgatcctgcttcttcatgcggtggtcttcagtctgccctacaccagggccaccgaggcctgtctgcgggcctgccctgcggcctgcacctgcagccacgtggaacgtggctgctcagtgcgctgtgaccgtgcgggcctccagcgggtgccccaggagtttccgtgcgaggcggcctccatcgatctggaccggaatggcctgcgcatcctgggcgagcgggcctttggcacgctgccgtcgttgcgccgcctgtcgctgcgccacaataacctgtccttcatcacgcccggcgccttcaagggcctgccgcggttggccgagctgcgcctggcgcacaacggtgagctgcgctacctgcacgtgcggaccttcgcggcgctgggccgcctacgccgcctggacctggcggcctgccgcctcttcagcgtccccgagcgtctcctggccgagctgccggccctgcgcgagctcacggccttcgacaatctcttccgccgggtgcccggcgcgctccggggcctcgccaacctgacgcacgctcatttcgagcgcagccgcatcgaggccgtggcctccggctcgctgctgggcatgcggcgtctgcgctcgctcagcctgcaggccaaccgcgtgcgcgcggtgcatgccggggcctttggcgactgcggcgccctggaggacctgctgctcaacgacaacctgctggccacgctgcccgccgccgccttccgcggccttcgccgcctgcgcaccctcaacctgggcggcaacgcgctgggcagcgtggcacgcgcctggttctcagacctggcagagctcgagctgctttacctggaccgcaacagcatcacctttgttgaggaaggcgccttccagaacctctcgggcctcctggccctgcatctcaatggcaaccgtctcactgtgctctcctgggccgctttccagccaggtttcttcctgggccgcctcttccttttccgcaatccttggcgctgtgactgccaactggagtggctgcgtgattggatggagggctctgggcgtgtggctgatgtggcgtgcgcctccccaggctctgtggccggccaggacctcagccaggtggtctttgagcgctcctctgatggcctctgtgtggaccctgatgaactgaactttaccacgtccagtcctggcccgagtccggagccagtggccaccactgtgagcaggttcagcagcctcctctccaagctgctggccccaagggcccctgtggaggaggtagccaataccacctgggagctggtcaacgtctcgttgaatgacagctttcggtcccatgcagtgatggtcttctgctacaaggccacgtttctcttcacctcttgcgtcttgctcagcctggcccagtatgtggtggtgggcctgcagagggagtga